(19) United States
(12) Patent Application Publication
(10) Pub. No.: US 2007/0053883 A1
(43) Pub. Date: Mar. 8, 2007

(54) Neuronal cell lineages and methods of production thereof

(75) Inventors: Philip A. Beachy, Stanford, CA (US); Michael K. Cooper, Nashville, TN (US)

Correspondence Address: DLA PIPER US LLP, 4365 EXECUTIVE DRIVE, SUITE 1100, SAN DIEGO, CA, 92121-2133, US

(73) Assignee: THE JOHNS HOPKINS UNIVERSITY, Baltimore, MD (US)

(21) Appl. No.: 10/550,220

(22) PCT Filed: Mar. 24, 2004
(86) PCT No.: PCT/US04/09051

§ 371 Date: Oct. 23, 2006

(30) Foreign Application Priority Data
Mar. 25, 2003 (US) 60457383

(51) Int. Cl. A61K 35/30; A61K 38/17; A61K 31/724; C12N 5/08

(52) U.S. Cl. 424/93.7; 435/368; 514/12; 514/58

(57) ABSTRACT

Methods are provided in which a sterol-depleting agent such as β-cyclodextrin (βCD), is used to modulate signaling by a differentiation signaling protein such as Hedgehog protein or a BMP in vitro. As illustrated herein, the use of βCD in conjunction with Sonic Hedgehog N-terminal peptide (ShhN) causes substantially all of the cells in neural plate explants to differentiate as neurons, such as motor neurons. Also provided are cell therapy methods and screening methods that rely on the modulation of signaling of a differentiation signaling protein such as Hedgehog, by a sterol-depleting agent such as βCD.

### BACKGROUND OF THE INVENTION

1. Field of the Invention

The invention relates generally to cell and developmental biology and more specifically to methods to induce differentiation in cells.

2. Background Information

Cell therapy holds great promise for reversing the ravages of degenerative disease by introducing healthy cells into a patient. Cell therapy has the potential to effectively treat virtually any disease in which normal cell function has been compromised. For example, cell therapy can be used to treat degenerative neurological diseases that have a major impact in society today, such as Parkinson's disease and ALS, as well as other important diseases such as diabetes.

The idea of cell therapy is based upon the ability to isolate or produce a particular cell type for use in replacing that cell in vivo. One of the more promising approaches to cell therapy is to produce the desired cells in any desired quantity by utilizing a stem or progenitor cell population that can be cultured and expanded in vitro. The problem is that differentiation of stem or progenitor cells often produces a limited number of cells of the desired type mixed with numerous cells of other distinct differentiated cell types. The ability to produce a uniform population of differentiated cells would increase yield and obviate the need to purify cells of the desired type, and would increase the effectiveness of the therapy.

Although methods have been presented for producing a particular cell type by stimulating stem or progenitor cells with factors in vitro, no such method produces a uniform population of differentiated cells, in particular in the field of neuronal differentiation. Current methods of producing differentiated neurons from stem cells result in production of a variety of distinct neuronal cell types. Production of a substantially uniform population of differentiated cell types would represent a significant improvement which would facilitate cell-based therapies in motor neuron diseases, Parkinson's disease, or more generally in other diseases in which deficits in a particular cell type occur and cell therapies are aimed at replacing that specific cell type.

### SUMMARY OF THE INVENTION

The present invention is based on the seminal discovery that contacting a stem or progenitor cell with a sterol-depleting agent and a differentiation signaling agent, results in differentiation of the stem or progenitor cell into a substantially uniform population of differentiated neurons. More particularly, it is shown that contact of stem cells and/or progenitor cells with β-cyclodextrin (βCD) and a Hedgehog protein results in differentiation of the stem and/or progenitor cells into a substantially uniform population of differentiated neurons. Therefore, the present invention provides a method to stimulate stem or progenitor cells with a signaling molecule in vitro to produce a uniform population of the desired differentiated cell type. With this approach it is possible to stimulate neural stem or progenitor cells with a differentiation signaling protein such as the Sonic Hedgehog protein and cause the entire population of cells to differentiate as a particular cell type such as motor neurons, or dopaminergic neurons. The method is particularly applicable to in vitro cell differentiation that involves the Hedgehog and TGF-β signaling pathways.

Accordingly, presented herein is a method of differentiating a population of stem cells or progenitor cells, including contacting the population of stem cells or progenitor cells with a Hedgehog protein under conditions sufficient to induce differentiation, and a β-cyclodextrin under conditions sufficient to decrease sterol concentrations in the population of cells and/or under conditions sufficient to positively effect TGFβ signaling in the population of cells, wherein the population of stem cells or progenitor cells differentiate into a substantially uniform population of differentiated cells. For example, the population of stem cells or progenitor cells can differentiate into a population of differentiated cells selected from cells of the central nervous system, intestinal cells, pancreatic cells, lung cells, and retinal cells. The population of stem cells or progenitor cells can be an isolated population of stem cells or progenitor cells, including an isolated population of neuronal stem cells or progenitor cells. The Hedgehog protein in certain aspects is a Sonic Hedgehog protein.

In another embodiment, a method for differentiating a stem cell or a progenitor cell into a neuron is included. The method includes contacting a stem cell or a progenitor cell with a differentiation signaling protein and a sterol-depleting agent under conditions sufficient to decrease sterol concentrations in the cell and/or under conditions sufficient to positively effect TGFβ signaling, wherein the differentiation signaling protein is selected from Hedgehog or a Transforming Growth Factor β (TGFβ) family member, until the stem cell or the progenitor cell differentiates into a neuron. The method can be used to differentiate a population of stem cells or a population of progenitor cells into a substantially uniform population of neurons.

In an illustrative embodiment, a method for differentiating a stem cell or a progenitor cell into a neuron is provided. The method includes contacting the stem cell or the progenitor cell with a Hedgehog protein and cyclodextrin (CD) under conditions sufficient to decrease sterol concentrations in the cell, until the stem cell or the progenitor cell differentiates into a neuron. Substantially uniform populations of differentiated cells can be introduce introduced into an animal, such as a mammal, in cell therapy methods provided herein. The Hedgehog protein in certain aspects is a Sonic Hedgehog protein.

In another embodiment, provided herein is a method to change the responsiveness of a stem cell or a progenitor cell to a Hedgehog signal, including contacting the stem cell or the progenitor cell with cyclodextrin (CD) in vitro under conditions sufficient to decrease sterol concentrations in the cell; and contacting the stem cell or the progenitor cell with a Hedgehog protein, thereby changing the responsiveness to a Hh signal. The method can further include detecting expression of a Hedgehog responsive gene and/or a gene whose is expression is associated with neuron differentiation.

In yet another embodiment, provided herein is a method for identifying a test compound that restores responsiveness to a Hedgehog (Hh) signal, including contacting a cell with a Hh protein, β-cyclodextrin (βCD), under conditions sufficient to decrease sterol concentrations in the cell; and a test compound. Test compounds that restore responsiveness to an Hh signal can be identified by identifying test compounds that stimulate a higher level of responsiveness to the Hh signal as compared with the level of responsiveness in the absence of the test compound.

### BRIEF DESCRIPTION OF DRAWINGS

FIGS. 1a-1b diagrammatically illustrate Cholesterol biosynthesis and an Hh pathway.

FIGS. 2a-2c illustrate inhibition of Shh signaling by cyclodextrin treatment inhibited. a, Cyclodextrin treatment of chick embryos in ovo caused holoprosencephaly. b, Cyclodextrin treatment inhibited the cellular response to Shh protein. c, Cyclodextrin treatment did not inhibit a BMP-mediated signaling event.

FIGS. 3a-b illustrate that cells defective in cholesterol biosynthesis do not respond to Shh. a, Shh autoprocessing proceeded to completion in Dhcr7^{-/-} and Sc5d^{-/-} MEFs at low cholesterol levels. b, Response of Dhcr7^{-/-} and Sc5d^{-/-} MEFs to Shh protein was inhibited at low cholesterol levels.

FIGS. 4a-4d illustrate inhibition of Hh signal response by sterol depletion in cells with intact cholesterol biosynthesis. Hh signal response was inhibited either by chronic cyclodextrin treatment (a) or by statin exposure after acute cyclodextrin treatment (b).

FIGS. 5a-5c illustrate that sterol depletion inhibits Shh signaling downstream of Ptch at the level of Smo.

### DETAILED DESCRIPTION OF THE INVENTION

The present invention is based on the use of a sterol-depleting agent such as β-cyclodextrin (βCD) to modulate signaling by differentiation signaling proteins such as Hedgehog proteins, Bone Morphogenic Proteins (BMPs), growth differentiation factors (GDFs), or other Transforming Growth Factor β (TGFβ) members in vitro. As illustrated herein, the use of βCD in conjunction with Hedgehog (e.g., Sonic Hh protein), causes a majority of the cells in neural plate explants to differentiate to neural cells, such as motor neurons.

Accordingly, presented herein is a method of differentiating a population of stem cells or progenitor cells, including contacting the population of stem cells or progenitor cells with a Hedgehog protein under conditions sufficient to induce differentiation, and a β-cyclodextrin under conditions sufficient to decrease sterol concentrations in the population of cells and/or under conditions sufficient to positively effect TGFβ signaling in the population of cells, wherein the population of stem cells or progenitor cells differentiate into a substantially uniform population of differentiated cells. For example, the population of stem cells or progenitor cells can differentiate into a population of differentiated cells selected from cells of the central nervous system, intestinal cells, pancreatic cells, lung cells, and retinal cells. The population of stem cells or progenitor cells can be an isolated population of stem cells or progenitor cells, including an isolated population of neuronal stem cells or progenitor cells. The Hedgehog protein in certain aspects is a Sonic Hedgehog protein.

In another embodiment, a method for differentiating a stem cell or a progenitor cell into a neuron is included. The method includes contacting a stem cell or a progenitor cell with a differentiation signaling protein and a sterol-depleting agent under conditions sufficient to decrease sterol concentrations in the cell and/or under conditions sufficient to positively effect TGFβ signaling, wherein the differentiation signaling protein is selected from Hedgehog or a Transforming Growth Factor β (TGFβ) family member, until the stem cell or the progenitor cell differentiates into a neuron. The method can be used to differentiate a population of stem cells or a population of progenitor cells into a substantially uniform population of neurons.

In an illustrative embodiment, a method for differentiating a stem cell or a progenitor cell into a neuron is provided. The method includes contacting the stem cell or the progenitor cell with a Hedgehog protein and cyclodextrin (CD) under conditions sufficient to decrease sterol concentrations in the cell, until the stem cell or the progenitor cell differentiates into a neuron. By contacting the stem cell or the progenitor cell with an Hh protein, an Hh response pathway is activated in the stem cell or the progenitor cell causing the stem cell or the progenitor cell, to differentiate.

In another embodiment, provided herein is a method to change the responsiveness of a stem cell or a progenitor cell to a Hedgehog signal, including contacting the stem cell or the progenitor cell with cyclodextrin (CD) in vitro under conditions sufficient to decrease sterol concentrations in the cell; and contacting the stem cell or the progenitor cell with a Hedgehog protein, thereby changing the responsiveness to a Hh signal. The method can further include detecting expression of a Hedgehog responsive gene and/or a gene whose is expression is associated with neuron differentiation.

A “differentiation signaling protein” is a protein that induces differentiation of a stem cell or a progenitor cell into a differentiated cell through a Hedgehog signaling pathway or a TGFβ signaling pathway. In illustrative examples of methods provided herein, the differentiation signaling protein that is used to induce differentiation is a Hedgehog protein. For example, the differentiation signaling protein can be an N-terminal Hedgehog polypeptide, such as a N-terminal Sonic Hedgehog polypeptide (ShhN).

The Hh protein is used at a concentration sufficient to induce differentiation in the stem cell or progenitor cell of a desired differentiated cells. Concentrations of Hh that induce differentiation are known in the art and depend, for example, on the cell type contacted with the Hh, the particular Hh protein used, and the desired differentiated cell type (See e.g., Ericson et al., Cell: 90, 169 (1997). For example, the Hh protein can be used at a concentration of 0.01 nM to 1 μM, or in more specific examples, 0.5 nM to 100 nM, or 1 nM to 50 nM. For differentiation of neural plate explants into motor neurons a concentration of greater than 2 nM can be employed in certain aspects of the invention to drive differentiation toward motor neuron formation (Ericson et al., 1997). As illustrated in the Examples herein, ShhN can be used at a concentration of 30 nM to induce cells in neural plate explants to differentiate as motor neurons. Furthermore, the Examples illustrate that in NIH3T3 fibroblasts, 4 nM ShhN activates the hedgehog pathway.

By “progenitor” it is meant an oligopotent or multipotent stem cell which is able to divide without limit and, under specific conditions, can produce daughter cells which terminally differentiate such as into neurons. These cells can be used for transplantation into a heterologous or autologous host. By “heterologous” is meant a host other than the animal from which the progenitor cells were originally derived. By autologous is meant the identical host from which the cells were originally derived.

“Stem cells” are pluripotent cells derived from pre-embryonic, embryonic, or fetal tissue at any time after fertilization, and have the characteristic of being capable under appropriate conditions of producing progeny of several different cell types that are derivatives of all of the three germinal layers (endoderm, mesoderm, and ectoderm), according to a standard art-accepted test, such as the ability to form a teratoma in 8-12 week old SCID mice. In certain aspects, the stem cells are capable of differentiating into neurons.

Included in the definition of stem cells are embryonic cells of various types, exemplified by human embryonic stem (hES) cells, described by Thomson et al. (Science 282:1145, 1998); embryonic stem cells from other primates, such as Rhesus stem cells (Thomson et al., Proc. Natl. Acad. Sci. USA 92:7844, 1995), marmoset stem cells (Thomson et al., Biol. Reprod. 55:254, 1996) and human embryonic germ (hEG) cells (Shamblott et al., Proc. Natl. Acad. Sci. USA 95:13726, 1998). Other types of pluripotent cells are also included in the term. Any cells of primate origin that are capable of producing progeny that are derivatives of all three germinal layers are included, regardless of whether they were derived from embryonic tissue, fetal tissue, or other sources.

Stem cell cultures are described as “undifferentiated” when a substantial proportion of stem cells and their derivatives in the population display morphological characteristics of undifferentiated cells, clearly distinguishing them from differentiated cells of embryo or adult origin. Undifferentiated stem cells are easily recognized by those skilled in the art, and typically appear in the two dimensions of a microscopic view in colonies of cells with high nuclear/cytoplasmic ratios and prominent nucleoli. It is understood that colonies of undifferentiated cells within the population will often be surrounded by neighboring cells that are differentiated. Nevertheless, the undifferentiated colonies persist when the population is cultured or passaged under appropriate conditions, and individual undifferentiated cells constitute a substantial proportion of the cell population. Cultures that are substantially undifferentiated contain at least 20% undifferentiated stem cells, and can contain at least 40%, 60%, or 80% in order of increasing preference.

As indicated herein, a “sterol-depleting agent” is an agent that reduces sterol levels of a cell. In certain illustrative aspects, the sterol-depleting agent is β-cyclodextrin (βCD). Other sterol-depleting agents that can be used in methods of the present invention include, for example, nystatin, or filipin.

Using a sterol-depleting agent, sterol levels are typically reduced to levels sufficient to inhibit responsiveness of the cell to an Hh signal, while providing sufficient sterols to allow Hh autoprocessing and Hh signaling. It will be understood that a target reduction in sterol levels is cell type dependent, and dependent on other factors, such as the concentration and type of differentiation signaling protein, such as the type of Hh protein, used to induce an Hh signal. After contact with the sterol-depleting agent, the cells differentiate into a uniform population of differentiated neurons.

In certain aspects, sterol levels are reduced to levels obtained in cells treated with βCD and lacking 7-hydrocholesterol reductase (Dhcr7) or lathosterol 5-desaturase (Sc5d) enzymes, or cells from human subjects afflicted with SLOS or lathosterolosis. For example, sterol levels, such as cholesterol levels, can be reduced by about 10%, 20%, 25%, 30%, 40%, 50%, 60%, 60%, or 75% by the sterol-depleting agent. In certain examples sterol levels are reduced to below 75 μg/ml, 60 μg/ml, 50 μg/ml, 30 μg/ml, 25 μg/ml, and in certain illustrative embodiments, below 40 μg/ml. As illustrated herein in primary fibroblasts, decrease in sterols by about 25% to about 30 μg/ml is sufficient to affect responsiveness to an Hh signal.

Various concentrations of the sterol-depleting agent such as βCD, can be used for the methods provided herein to achieve adequate sterol level reductions. For example, the sterol-depleting agent is used at a concentration effective for reducing sterol levels to below 100 μg/mg, typically below 10 μg/mg.

The sterol-depleting agent such as βCD can be used continuously, or transiently. When βCD is used transiently, it is typically followed by continuous treatment with a sterol synthesis inhibitor for at least a portion of the time, and typically the entire time that a cell is exposed to a differentiation signal, such as an Hh signal. The sterol synthesis inhibitor is used at a concentration that is effective for at least partially blocking sterol synthesis, and typically for blocking the rate of sterol synthesis sufficiently such that sterol levels remain low enough after treatment with a sterol-depleting agent, to affect a differentiation signal, such as an Hh signal. In certain aspects, the sterol synthesis inhibitor blocks upstream of 3-hydroxy-3-methyl-glutaryl coenzyme A (HMG CoA) reductase in a sterol synthesis pathway, or blocks 3-hydroxy-3-methyl-glutaryl coenzyme A (HMG CoA) reductase. Accordingly, in one example, the sterol synthesis inhibitor is a statin. For example, the sterol synthesis inhibitor can be atorvastatin, fluvastatin, lovastatin, pravastatin, simvastatin, or compactin (i.e. mevastatin). Statins inhibit HMG CoA reductase-mediated conversion of HMG CoA to mevalonic acid, an early precursor of cholesterol.

When the sterol-depleting agent such as βCD is used in a transient manner, it can be applied to cells for between about 1 minute and 24 hours, for example between about 30 minutes and 12 hours, between about 1 hour and 4 hours, and in one illustrative example is treated for about 2 hours. In another example, transient βCD treatment is carried out for 5 minutes to 120 minutes, 15 minutes to 60 minutes, or in an illustrative example, for about 30 minutes. In transient treatment methods, the sterol-depleting agent is typically applied at the same time as, or before, a differentiation signal is elicited in cells.

When it is used continuously, for example, βCD can be used at a concentration of between about 1 μM and about 5 mM, more particularly between about 10 μM and about 1 mM, between 200 μM and 600 μM, between 300 μM and 500 μM, and more specifically. In an illustrative embodiment, using neural explant cells and continuous exposure, βCD can be used at a concentration greater than 200 μM, for example, 300 μM to 1 mM, or at about 400 μM. As indicated above, βCD is typically used at a higher concentration when it is used to transiently deplete a cell at least partially of sterols. For example, when used transiently, the βCD can be used for example at 0.5 to 50 mM, 1 to 10 mM, or in one example, at about 3.8 mM embryonic fibroblasts are analyzed.

A “Hedgehog responsive gene” is a gene whose expression is affected by Hh signaling. Hh responsive genes are known in the art. For example, Gli and Patched (Ptc) are Hh responsive genes. As illustrated herein, a reporter can be rendered Hh responsive by operatively linking the reporter to a Gli response element.

A method of the invention is performed, for example, by contacting a stem cell or a progenitor cell with a βCD. As used herein, the term “contacting,” when used in reference to a βCD and a stem cell or a progenitor cell, means that the βCD is placed in sufficient proximity to the stem cell or the progenitor cell such that it reduces sterol concentrations in the cell and/or positively effects TGFβ, for example BMP, signaling.

In the methods provided herein, the stem cell or the progenitor cell are contacted with βCD or another sterol-depleting agent under conditions sufficient to decrease sterol concentrations and/or to induce signaling by TGFβ family members, such as BMPs. It will be understood that such conditions will vary depending on the particular stem cell and progenitor cell. Furthermore, it will be understood that these conditions include, for example, time contacted by a differentiation signaling protein and by a sterol-depleting agent, temperature, concentration and specific differentiation signaling protein and steroldepleting agent, and order of contact by the differentiation signaling protein and the sterol-depleting agent.

Regarding the order of contact, cells can be contacted by the sterol-depleting agent before being contacted by a differentiation signaling protein, or for at least a portion of the time that the cells are contacted with the differentiation signaling protein. Alternatively, as illustrated in the Examples herein, the cells can be contacted with the differentiation signaling protein (e.g., ShhN) before and while being contacted with the sterol-depleting agent (e.g., βCD). In certain examples, cells are contacted by the sterol-depleting agent acutely for an effective period of time (e.g., 1, 2, 5, 10, 15, 30, 45, 60, 120, or 180 minutes), and then the sterol-depleting agent is removed from the cells and the cells are incubated such that sterol levels remain reduced. For example, after withdrawal from contact with βCD, cells can be cultured in a lipid-depleted medium, such as lipid depleted serum, and/or in the presence of a sterol-perturbation agent, such as a compactin. Alternatively, the cells can have a genetic defect in sterol biosynthesis.

As illustrated in the Examples provided herein, the βCD is typically added in a liquid form to cells in culture, thereby contacting the cells with the βCD. The stem cell or the progenitor cell, typically a population of stem cells or progenitor cells in culture, remain in contact with βCD for between about 5 minutes and about 7 days. For example, the cells can remain in contact with βCD for between about 15 minutes and about 72 hours. In the example provided herein, neural plate cells were contacted with βCD for 48 hours. During and optionally after contact with βCD, the cells are typically cultured in lipid-depleted serum.

Stem cells or progenitor cells undergo differentiation in certain exemplary methods provided herein, substantially synchronously. “Substantially synchronously” means that cells attain a differentiated state within about 48 hours, more typically within 24 hours, and in certain illustrative examples, within 12, 8, 4, 2, 1, 0.5, 0.25, or 0.1 hours of each other. In certain aspects, the stem cells are neuronal stem cells and/or isolated stem cells.

As mentioned above and illustrated herein, the use of CD in conjunction with a Hedgehog protein causes virtually all of the cells in a population of stem cells or progenitor cells to undergo differentiation into differentiated neurons. The population of isolated cells is typically a culture of the cells. Accordingly, in certain examples, a population of stem cells or progenitor cells are contacted with a Sonic Hedgehog protein and cyclodextrin (CD) under conditions sufficient to decrease sterol concentrations in the cells, until a majority of cells differentiate into neurons. In certain illustrative examples, at least 75%, 80%, 85%, 90%, 95%, 99%, 99.5%, or 100% of the stem cells or progenitor cells undergo differentiation into differentiated neurons in the culture. In other examples, a population of stem cells or progenitor cells in a culture undergo differentiation to form a substantially uniform population of neurons. A “substantially uniform population” of cells means that at least 90% of the cells in the population are differentiated neurons.

Accordingly, in certain embodiments, provided herein is a population of differentiated neurons produced by the in vitro method disclosed herein. For example, at least 75%, 80%, 85%, 90%, 95%, 99%, 99.5%, or 100% of the stem cells or progenitor cells in the population of cells, are in a differentiated state. In certain examples, the population of differentiated neurons are a substantially uniform population of differentiated neurons. A substantially uniform population, is a population wherein at least 90% of the cells in the population are differentiated cells, such as neurons.

In methods provided herein, the stem cells and/or progenitor cells are contacted with a Hedgehog protein or a TGFβ family member, such as a BMP, by adding a Hedgehog protein or a TGFβ family member, such as a BMP, to a culture medium in which the stem cells and/or progenitor cells are maintained. Alternatively, the Hedgehog protein or TGFβ family member can be supplied in conditioned medium, harvested from cultures of cells that excrete these proteins. The Hedgehog protein or TGFβ family member is typically provided in the culture medium at a concentration effective for inducing differentiation, as disclosed herein.

As indicated above, in addition to the negative effect of βCD treatment (and consequent cholesterol depletion) upon Shh signaling, the role of βCD in inducing differentiation when used to contact a cell along with a differentiation signaling protein may be due to the positive effect of βCD treatment upon signaling by BMP proteins (TGFβ family members) (Di Guglielmo et al., 2003). Accordingly, in certain examples, the stem cell or the progenitor cell is contacted with βCD under conditions sufficient to positively affect TGFβ signaling. More particularly, the cell can be contacted with βCD under conditions sufficient for positively affecting signaling by BMPs. In these aspects, the stem cell or the progenitor cell is contacted with a Bone Morphogenic Protein (BMP) to induce differentiation. For example BMP can be used, for example, at 0.1 to 5.0 units.

As indicated herein, and not intended to be limited by a particular theory, contact of a population of stem cells with a sterol-depleting agent such as βCD is believed to be useful in methods provided herein because the βCD is believed to bring substantially all, or all of the stem cells and progenitor cells in a culture to a similar state of responsiveness to a Hedgehog protein signal and/or a TGFβ signal, thus enhancing the uniformity of a differentiation response upon exposure of the cultured cells to a Hedgehog protein or a TGFβ family member, such as a BMP. The Hh protein or TGFβ family member, such as a BMP, is typically added to the culture medium.

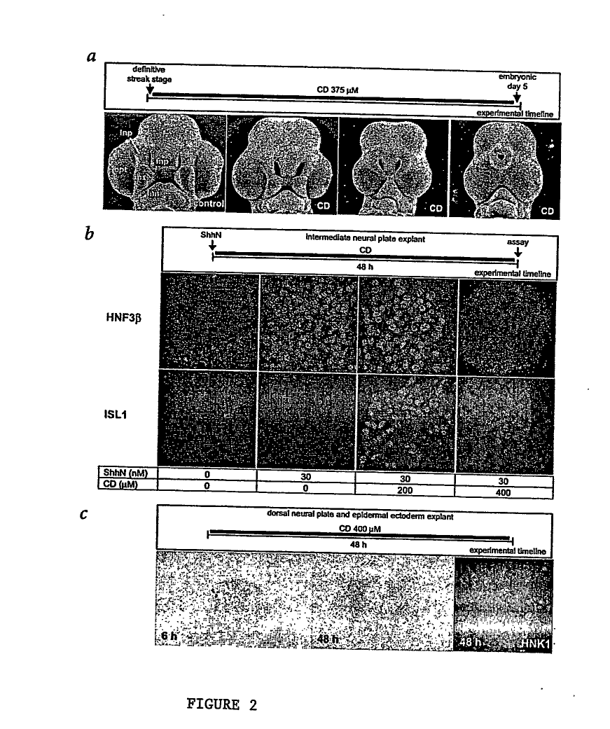

It will be understood that several independent criteria can be used to identify differentiated neurons in culture. These include morphological characteristics during growth, expression of neuronal markers and ultrastructural analysis by transmission and scanning electron microscopy. Cell morphology in culture has been reported for short-term cultures of neurons (Banker and Cowan, Brain Res., 126:397-425, 1977; Banker and Cowan, J. Comp. Neurol., 187:469-494,1979) (FIGS. 2A, B, C). Cells are typically larger and interconnected by processes that also increase in size. Cells can also be identified as neurons by immunostaining them for several different antigenic markers. For example, cells can be stained with anti-NF (200 KD) antibody or with anti-NSE antibody.

Genes whose expression is associated with neuron differentiation are also known in the art and can be detected in order to identify a cell as a differentiated neuron. As exemplified herein these genes include Isl1.

Bone morphogenetic proteins (BMP) induce ectopic bone formation, and plays an important role in the development of the viscera. Any bone morphogenic protein is contemplated including bone morphogenic proteins designated as BMP-1 through BMP-13. The BMP can be a recombinant BMP, such as a recombinant human BMP. The BMP can be a mammalian BMP, such as a human BMP. BMPs are commercially available from Genetics Institute, Inc., Cambridge, Mass. and may also be prepared by one skilled in the art as described in U.S. Pat. No. 5,187,076 to Wozney et al.; U.S. Pat. No. 5,366,875 to Wozney et al.; U.S. Pat. No. 4,877,864 to Wang et al.; U.S. Pat. No. 5,108,922 to Wang et al.; U.S. Pat. No. 5,116,738 to Wang et al.; U.S. Pat. No. 5,013,649 to Wang et al.; U.S. Pat. No. 5,106,748 to Wozney et al.; and PCT Patent Nos. WO93/00432 to Wozney et al.; WO94/26893 to Celeste et al.; and WO94/26892 to Celeste et al

According to the present invention, large numbers of neural progenitor cells are perpetuated in vitro and their differentiation occurs in a substantially uniform manner by contact with a sterol-depleting agent such as βCD, along with a differentiation signaling protein such as ShhN. A method can include the steps of isolating neural progenitor cells from an animal, perpetuating these cells in vitro or in vivo, preferably in the presence of growth factors, and regulating the differentiation of these cells into particular neural phenotypes, e.g., neurons, by contacting the cells with a differentiation signaling protein, such as ShhN and a sterol-depleting agent such as βCD.

Progenitor cells are thought to be under a tonic inhibitory influence that maintains the progenitors in a suppressed state until their differentiation is required. However, recent techniques have been provided which permit these cells to be proliferated, and unlike neurons which are terminally differentiated and therefore non-dividing, they can be produced in unlimited number and are highly suitable for transplantation into heterologous and autologous hosts with neurodegenerative diseases.

Cells can be obtained from embryonic, post-natal, juvenile or adult neural tissue from any animal. By “any animal” is meant any multicellular animal which contains nervous tissue. More particularly, is meant any fish, reptile, bird (e.g. chicken), amphibian or mammal and the like. Illustrative donors include mammals, such as mice and humans. The example provided herein demonstrates the present invention using chicken neural plate explants. However, it is known that both Hh, Hh processing, and Hh response pathways are well conserved from Drosophila through various vertebrate species, including mammalian species such as humans. Accordingly, it will be understood that the examples provided herein in chicken cells are illustrative and can be extrapolated to other vertebrate species including humans.

In the case of a heterologous donor animal, the animal can be euthanized, and the brain and specific area of interest removed using a sterile procedure. Brain areas of particular interest include any area from which progenitor cells can be obtained which will serve to restore function to a degenerated area of the host's brain. These regions include areas of the central nervous system (CNS) including the cerebral cortex, cerebellum, midbrain, brainstem, spinal cord and ventricular tissue, and areas of the peripheral nervous system (PNS) including the carotid body and the adrenal medulla. More particularly, these areas include regions in the basal ganglia, preferably the striatum which consists of the caudate and putamen, or various cell groups such as the globus pallidus, the subthalamic nucleus, the nucleus basalis which is found to be degenerated in Alzheimer's Disease patients, or the substantia nigra pars compacta which is found to be degenerated in Parkinson's Disease patients.

Human heterologous neural progenitor cells can be derived from fetal tissue obtained from elective abortion, or from a post-natal, juvenile or adult organ donor. Autologous neural tissue can be obtained by biopsy, or from patients undergoing neurosurgery in which neural tissue is removed, in particular during epilepsy surgery, and more particularly during temporal lobectomies and hippocampalectomies.

Cells can be obtained from donor tissue by dissociation of individual cells from the connecting extracellular matrix of the tissue. Dissociation can be obtained using any known procedure, including treatment with enzymes such as trypsin, collagenase and the like, or by using physical methods of dissociation such as with a blunt instrument or by mincing with a scalpel to a allow outgrowth of specific cell types from a tissue. Dissociation of fetal cells can be carried out in tissue culture medium, while a preferable medium for dissociation of juvenile and adult cells is artificial cerebral spinal fluid (aCSF). Regular aCSF contains 124 mM NaCl, 5 mM KCl, 1.3 mM MgCl₂, 2 mM CaCl₂, 26 mM NaHCO₃, and 10 mM D-glucose. Low Ca²⁺ aCSF contains the same ingredients except for MgCl₂ at a concentration of 3.2 mM and CaCl₂ at a concentration of 0.1 mM.

Dissociated cells can be placed into any known culture medium capable of supporting cell growth, including MEM, DMEM, RPMI, F-12, and the like, containing supplements which are required for cellular metabolism such as glutamine and other amino acids, vitamins, minerals and useful proteins such as transferrin and the like. Medium may also contain antibiotics to prevent contamination with yeast, bacteria and fungi such as penicillin, streptomycin, gentamicin and the like. In some cases, the medium may contain serum derived from bovine, equine, chicken and the like. A particularly preferable medium for cells is a mixture of DMEM and F-12. Furthermore, the medium can be depleted of lipids using known methods, as disclosed in further detail herein.

Conditions for culturing should be close to physiological conditions. The pH of the culture media should be close to physiological pH, for example between pH 6-8, even more particularly about pH 7.4. Cells should be cultured at a temperature close to physiological temperature, preferably between 30° C.-40° C., more preferably between 32° C.-38° C., and most preferably between 35° C.-37° C.

Cells can be grown in suspension or on a fixed substrate, but proliferation of the progenitors is preferably done in suspension to generate large numbers of cells by formation of “neurospheres” (see, for example, Reynolds et al. (1992) Science 255:1070-1709; and PCT Publications WO93/01275, WO94/09119, WO94/10292, and WO94/16718). In the case of propagating (or splitting) suspension cells, flasks are shaken well and the neurospheres allowed to settle on the bottom corner of the flask. The spheres are then transferred to a 50 ml centrifuge tube and centrifuged at low speed. The medium is aspirated, the cells resuspended in a small amount of medium with growth factor, and the cells mechanically dissociated and resuspended in separate aliquots of media.

Cell suspensions in culture medium are supplemented with any growth factor which allows for the proliferation of progenitor cells and seeded in any receptacle capable of sustaining cells, though as set out above, preferably in culture flasks or roller bottles. Cells typically proliferate within 3-4 days in a 37° C. incubator, and proliferation can be reinitiated at any time after that by dissociation of the cells and resuspension in fresh medium containing growth factors.

In the absence of substrate, cells lift off the floor of the flask and continue to proliferate in suspension forming a hollow sphere of undifferentiated cells. After approximately 3-10 days in vitro, the proliferating clusters (neurospheres) are fed every 2-7 days, and more particularly every 2-4 days by gentle centrifugation and resuspension in medium containing growth factor. After 6-7 days in vitro, individual cells in the neurospheres can be separated by physical dissociation of the neurospheres with a blunt instrument, more particularly by triturating the neurospheres with a pipette. Single cells from the dissociated neurospheres are suspended in culture medium containing growth factors, and differentiation of the cells can be controlled in culture by plating (or resuspending) the cells in the presence of a differentiation signaling protein and a sterol-depleting agent.

Stem cells useful in methods provided herein are generally known. For example, several neural crest cells have been identified, some of which are multipotent and likely represent uncommitted neural crest cells, and others of which can generate only one type of cell, such as sensory neurons, and likely represent committed progenitor cells. The role of the differentiation signaling protein and the sterol-depleting agent in methods provided herein can be to regulate differentiation of the uncommitted progenitor, or to regulate further restriction of the developmental fate of a committed progenitor cell towards becoming a terminally differentiated neuronal cell. For example, the present method can be used in vitro to regulate the differentiation of neural stem cells into motor neurons, dopaminergic neurons, glial cells, schwann cells, chromaffin cells, cholinergic sympathetic or parasympathetic neurons, as well as peptidergic and serotonergic neurons. In other illustrative examples, methods of the present invention are used to differentiate neural plate explants into motor neurons. Methods are known for identifying a differentiated cell such as a glial cell, schwann cell, chromaffin cell, cholinergic sympathetic or parasympathetic neuron, as well as peptidergic and serotonergic neuron. The methods provided herein can further include contacting the stem cell or the progenitor cell with other neurotrophic factors which act to more particularly enhance a particular differentiation fate of the progenitor cell.

The vertebrate family of Hedgehog proteins includes at least four members, any of which can be used in methods provided herein. Exemplary Hedgehog proteins are described in PCT publications WO 95/18856 and WO 96/17924. Three of these members, herein referred to as Desert Hedgehog (Dhh), Sonic Hedgehog (Shh) and Indian Hedgehog (Ihh), apparently exist in all vertebrates, including fish, birds, and mammals. A fourth member, herein referred to as tiggie-winkle Hedgehog (Thh), appears specific to fish. Desert Hedgehog (Dhh) is expressed principally in the testes, both in mouse embryonic development and in the adult rodent and human; Indian Hedgehog (Ihh) is involved in bone development during embryogenesis and in bone formation in the adult; and, Shh, which is primarily involved in morphogenic and neuroinductive activities. Given the critical inductive roles of Hedgehog polypeptides in the development and maintenance of vertebrate organs, the identification of agents that can be used to bring substantially all cells in a culture to an identical Hedgehog response state, is of paramount significance in both clinical and research contexts.

The various Hedgehog proteins consist of a signal peptide, a highly conserved N-terminal region, and a more divergent C-terminal domain. In addition to signal sequence cleavage in the secretory pathway (Lee, J. J. et al. (1992) Cell 71:33-50; Tabata, T. et al. (1992) Genes Dev. 2635-2645; Chang, D. E. et al. (1994) Development 120:3339-3353), Hedgehog precursor proteins undergo an internal autoproteolytic cleavage which depends on conserved sequences in the C-terminal portion (Lee et al. (1994) Science 266:1528-1537; Porter et al. (1995) Nature 374:363-366). This autocleavage leads to a 19 kD N-terminal peptide and a C-terminal peptide of 26-28 kD (Lee et al. (1992) supra; Tabata et al. (1992) supra; Chang et al. (1994) supra; Lee et al. (1994) supra; Bumcrot, D. A., et al. (1995) Mol. Cell. Biol. 15:2294-2303; Porter et al. (1995) supra; Ekker, S. C. et al. (1995) Curr. Biol. 5:944-955; Lai, C. J. et al. (1995) Development 121:2349-2360). The N-terminal peptide, for example a sonic N-terminal peptide provides a differentiation signal in certain illustrative aspects of methods provided herein. The N-terminal peptide stays tightly associated with the surface of cells in which it was synthesized, while the C-terminal peptide is freely diffusible both in vitro and in vivo (Porter et al. (1995) Nature 374:363; Lee et al. (1994) supra; Bumcrot et al. (1995) supra; Mart', E. et al. (1995) Development 121:2537-2547; Roelink, H. et al. (1995) Cell 81:445-455). Interestingly, cell surface retention of the N-terminal peptide is dependent on autocleavage, as a truncated form of Hh encoded by an RNA which terminates precisely at the normal position of internal cleavage is diffusible in vitro (Porter et al. (1995) supra) and in vivo (Porter, J. A. et al. (1996) Cell 86, 21-34). Biochemical studies have shown that the autoproteolytic cleavage of the Hh precursor protein proceeds through an internal thioester intermediate which subsequently is cleaved in a nucleophilic substitution. It is likely that the nucleophile is a small lipophilic molecule which becomes covalently bound to the C-terminal end of the N-peptide (Porter et al. (1996) supra), tethering it to the cell surface. The biological implications are profound. As a result of the tethering, a high local concentration of N-terminal Hedgehog peptide is generated on the surface of the Hedgehog producing cells. It is this N-terminal peptide which is both necessary and sufficient for short- and long-range Hedgehog signaling activities in Drosophila and vertebrates (Porter et al. (1995) supra; Ekker et al. (1995) supra: Lai et al. (1995) supra; Roelink, H. et al. (1995) Cell 81:445-455; Porter et al. (1996) supra; Fietz, M. J. et al. (1995) Curr. Biol. 5:643-651; Fan, C.-M. et al. (1995) Cell 81:457-465; Mart', E., et al. (1995) Nature 375:322-325; Lopez-Martinez et al. (1995) Curr. Biol 5:791-795; Ekker, S. C. et al. (1995) Development 121:2337-2347; Forbes, A. J. et al. (1996) Development 122:1125-1135). Accordingly, in certain illustrative embodiments of the methods provided herein, the Hedgehog protein is an N-terminal Hedgehog peptide.

Hh has been implicated in short- and long-range patterning processes at various sites during Drosophila development. In the establishment of segment polarity in early embryos, it has short-range effects which appear to be directly mediated, while in the patterning of the imaginal discs, it induces long range effects via the induction of secondary signals.

In vertebrates, several Hedgehog genes have been cloned. Of these genes, Shh has received most of the experimental attention, as it is expressed in different organizing centers which are the sources of signals that pattern neighboring tissues. Recent evidence indicates that Shh is involved in these interactions.

The expression of Shh starts shortly after the onset of gastrulation in the presumptive midline mesoderm, the node in the mouse (Chang et al. (1994) supra; Echelard, Y. et al. (1993) Cell 75:1417-1430), the rat (Roelinlc, H. et al. (1994) Cell 76:761-775) and the chick (Riddle, R. D. et al. (1993) Cell 75:1401-1416), and the shield in the zebrafish (Ekker et al. (1995) supra; Krauss, S. et al. (1993) Cell 75:1431-1444). In chick embyros, the Shh expression pattern in the node develops a left-right asymmetry, which appears to be responsible for the left-right situs of the heart (Levin, M. et al. (1995) Cell 82:803-814).

In the CNS, Shh from the notochord and the floorplate appears to induce ventral cell fates. When ectopically expressed, Shh leads to a ventralization of large regions of the mid- and hindbrain in mouse (Echelard et al. (1993) supra; Goodrich, L. V. et al. (1996) Genes Dev. 10:301-312), Xenopus (Roelink, H. et al. (1994) supra; Ruiz i Altaba, A. et al. (1995) Mol. Cell. Neurosci. 6:106-121), and zebrafish (Ekker et al. (1995) supra; Krauss et al. (1993) supra; Hammerschmidt, M., et al. (1996) Genes Dev. 10:647-658). In explants of intermediate neuroectoderm at spinal cord levels, Shh protein induces Doorplate and motor neuron development with distinct concentration thresholds, floor plate at high and motor neurons at lower concentrations (Roelink et al. (1995) supra; Mart' et al. (1995) supra; Tanabe, Y. et al. (1995) Curr. Biol. 5:651-658). Moreover, antibody blocking suggests that Shh produced by the notochord is required for notochord-mediated induction of motor neuron fates (Mart' et al. (1995) supra). Thus, high concentration of Shh on the surface of Shh-producing midline cells appears to account for the contact-mediated induction of floorplate observed in vitro (Placzek, M. et al. (1993) Development 117:205-218), and the midline positioning of the floorplate immediately above the notochord in vivo. Lower concentrations of Shh released from the notochord and the floorplate presumably induce motor neurons at more distant ventrolateral regions in a process that has been shown to be contact-independent in vitro (Yamada, T. et al. (1993) Cell 73:673-686). In explants taken at midbrain and forebrain levels, Shh also induces the appropriate ventrolateral neuronal cell types, dopaminergic (Heynes, M. et al. (1995) Neuron 15:35-44; Wang, M. Z. et al. (1995) Nature Med. 1:1184-1188) and cholinergic Ericson, J. et al. (1995) Cell 81:747-756) precursors, respectively, indicating that Shh is a common inducer of ventral specification over the entire length of the CNS.

Shh from the midline also patterns the paraxial regions of the vertebrate embryo, the somites in the trunk (Fan et al. (1995) supra) and the head mesenchyme rostral of the somites (Hammerschmidt et al. (1996) supra). In chick and mouse paraxial mesoderm explants, Shh promotes the expression of sclerotome specific markers like Pax1 and Twist, at the expense of the dermamyotomal marker Pax3. Moreover, filter barrier experiments suggest that Shh mediates the induction of the sclerotome directly rather than by activation of a secondary signaling mechanism (Fan, C.-M. and Tessier-Lavigne, M. (1994) Cell 79, 1175-1186).

Shh in the vertebrate limb bud activates the expression of Bmp2 (Francis, P. H. et al. (1994) Development 120:209-218), a dpp homologue. However, unlike DPP in Drosophila, Bmp2 fails to mimic the polarizing effect of Shh upon ectopic application in the chick limb bud (Francis et al. (1994) supra). In addition to anteroposterior patterning, Shh also appears to be involved in the regulation of the proximodistal outgrowth of the limbs by inducing the synthesis of the fibroblast growth factor FGF4 in the posterior apical ectodermal ridge (Laufer, E. et al. (1994) Cell 79:993-1003; Niswander, L. et al. (1994) Nature 371:609-612).

The close relationship between Hedgehog proteins and BMPs is likely to have been conserved at many, but probably not all sites of vertebrate Hedgehog expression. For example, in the chick hindgut, Shh has been shown to induce the expression of Bmp4, another vertebrate dpp homologue (Roberts, D. J. et al. (1995) Development 121:3163-3174). Furthermore, Shh and Bmp2, 4, or 6 show a striking correlation in their expression in epithelial and mesenchymal cells of the stomach, the urogential system, the lung, the tooth buds and the hair follicles (Bitgood, M. J. and McMahon, A. P. (1995) Dev. Biol. 172:126-138). Further, Ihh, one of the two other mouse Hedgehog genes, is expressed adjacent to Bmp expressing cells in the gut and developing cartilage (Bitgood and McMahon (1995) supra).

Recent evidence suggests a model in which Indian Hedgehog (Ihh) plays a crucial role in the regulation of chondrogenic development (Roberts et al. (1995) supra). In certain aspects, provided herein is a method for obtaining a substantially uniform population of chondrocytes by contacting stem cells including proliferating chondrocytes with a Hh protein, such as an Ihh protein. In other aspects, a population of stem cells that are progenitor immune cells are contacted with βCD and a Hh protein to obtain a substantially uniform population of differentiated immune cells.

In certain aspects, the sterol-depleting agent is β-cyclodextrin (βCD). The methods of the invention are exemplified using β-methyl cyclodextrin. As illustrated herein, the use of 400 μM β-methyl CD in conjunction with 30 nM ShhN caused all cells in neural plate explants to differentiate as motor neurons. Virtually any βCD derivative can be used in a method of the invention, provided the βCD derivative reduces sterol concentrations in the cell and/or positively effects TGFβ signaling. Furthermore, βCDs act, at least in part, by removing cholesterol from cell membranes. Different βCDs are variably effective in such removal. For example, methyl-βCD is particularly effective at removing cholesterol from cell membranes. Thus, it will be recognized that a βCD useful in a method of the invention can be one that decreases sterol concentrations of cells, for example by removing cholesterol from cell membranes.

Not intended to be limited by a particular theory, in addition to the negative effect of βCD treatment (and consequent cholesterol depletion) upon response to an Hh signal, the role of βCD in inducing differentiation when used to contact a cell along with a differentiation signaling protein may be due to the positive effect of βCD treatment upon signaling by BMP proteins (TGFβ family members) (Di Guglielmo et al., 2003). In developing neurons within the embryonic neural tube, the determination of differentiated cell type depends upon the levels of Hh and BMP signaling, as these signals represent ventral and dorsal patterning influences, respectively, and oppose each other's actions (Patten and Placzek, 2002; Liem et al. 2003). The use of βCD is believe to bring all cells to a similar state of responsiveness to both a Hedgehog and a TGFβ signal, thus enhancing the uniformity of response.

β-cyclodextrins (βCDs) are widely used as solubilizing agents, stabilizers, and inert excipients in pharmaceutical compositions (see U.S. Pat. Nos. 6,194,430; 6,194,395; and 6,191,137, each of which is incorporated herein by reference). βCDs are cyclic compounds containing seven units of α-(1→4) linked D-glucopyranose units, and act as complexing agents that can form inclusion complexes and have concomitant solubilizing properties (see U.S. Pat. No. 6,194,395; see, also, Szejtli, J. Cyclodextrin Technol. 1988).

βCDs useful in the present invention include, for example, βCD derivatives wherein one or more of the hydroxy groups is substituted by an alkyl, hydroxyalkyl, carboxyalkyl, alkylcarbonyl, carboxyalkoxyalkyl, alkylcarbonyloxyalkyl, alkoxycarbonylalkyl or hydroxy-(mono or polyalkoxy)alkyl group or the like; and wherein each alkyl or alkylene moiety contains up to about six carbons. Substituted βCDs that can be used in the present invention include, for example, polyethers (see, for example, U.S. Pat. No. 3,459,731, which is incorporated herein by reference); ethers, wherein the hydrogen of one or more βCD hydroxy groups is replaced by C1 to C6 alkyl, hydroxy-C1-C6-alkyl, carboxy-C1-C6 alkyl, C1-C6 alkyloxycarbonyl-C1-C6 alkyl groups, or mixed ethers thereof. In such substituted βCDs, the hydrogen of one or more βCD hydroxy group can be replaced by C1-C3 alkyl, hydroxy-C2-C4 alkyl, or carboxy-C1-C2 alkyl, for example, by methyl, ethyl, hydroxyethyl, hydroxypropyl, hydroxybutyl, carboxymethyl or carboxyethyl. It should be recognized that the term “C1-C6 alkyl” includes straight and branched saturated hydrocarbon radicals, having from 1 to 6 carbon atoms. Examples of βCD ethers include dimethyl-βCD. Examples of βCD polyethers include hydroxypropyl-p-βCD and hydroxyethyl-βCD (see, for example, Nogradi, “Drugs of the Future” 9(8):577-578, 1984; Chemical and Pharmaceutical Bulletin 28: 1552-1558, 1980; Yakugyo Jiho No. 6452 (28 Mar. 1983); Angew. Chem. Int. Ed. Engl. 19: 344-362, 1980; U.S. Pat. No. 3,459,731; EP-A-0,149,197; EP-A-0,197,571; U.S. Pat. No. 4,535,152; WO-90/12035; GB-2,189,245; Szejtli, “Cyclodextrin Technology” (Kluwer Academic Publ. 1988); Bender et al., “Cyclodextrin Chemistry” (Springer-Verlag, Berlin 1978); French, Adv. Carb. Chem. 12:189-260; Croft and Bartsch, Tetrahedron 39:1417-1474, 1983; Irie et al., Pharm. Res. 5:713-716, 1988; Pitha et al., Internat'l. J. Pharm. 29:73, 1986; U.S. Pat. No. 5,134,127 A; U.S. Pat. Nos. 4,659,696 and 4,383,992, each of which is incorporated herein by reference; see, also, U.S. Pat. No. 6,194,395). Another βCD of that can be used in the methods of the invention is 2-hydroxypropyl-βCD (2-OH—βCD).

The methods to differentiate a stem cell or progenitor cell, provided herein, are useful, for example, for production of differentiated cells in vitro that can be used, for example, for cell therapy. Such therapies are applicable in diseases such as Parkinson's, ALS, and other degenerative neurological diseases, or in diabetes or any other disease relating to deficiency of a particular cell type. The methods herein can be used to provide a population of differentiated cells that can be introduced into a subject in need of such cells.

Accordingly, provided herein is a method to introduce a cell into an animal, such as a mammal, including: differentiating a stem cell or a progenitor cell into a differentiated cell in vitro by the methods for differentiating stem cells or progenitor cells provided herein, and introducing the differentiated cell into the animal. Typically, the stem cell or progenitor cell is a member of a population of stem cells or progenitor cells that are differentiated into a population of substantially uniform differentiated cells according to methods provided herein. The animal can be a mammal, such as a human. In certain aspects, the population of stem cells or progenitor cells differentiate into a population of differentiated cells selected from cells of the central nervous system, intestinal cells, pancreatic cells, lung cells, and cells of the retina.

In an illustrative aspect, the stem cell population or the progenitor cell population is a stem cell population or a progenitor cell population. In this aspect, the differentiated cell population is a substantially uniform population of neurons, such as a motor neurons or dopaminergic neurons. The animal can be, for example, a mammal, such as a human, afflicted with a neurodegenerative disorder. Exemplary neurodegenerative disorders include Parkinson's Disease and ALS.

In examples wherein the substantially uniform differentiated population of cells are pancreatic cells, the method herein can be used to treat disorders involving pancreatic cells, such as diabetes. In other examples, wherein the substantially uniform differentiated population of cells are cells of the retina, the method herein can be used to treat degenerative disorders of the retina.

The populations of differentiated cells of the subject invention can be administered as cell therapy to alleviate the symptoms of a wide variety of disease states and pathological conditions, in various stages of pathological development. For example, differentiated cells provided herein, or stem cells that have been contacted with a sterol-depleting agent can be used to treat chronic disorders (e.g., Parkinson's disease, ALS, diabetes, or muscular dystrophy), and administered preventatively and/or prophylactically, early in the disease state, in moderate disease states, or in severe disease states. For example, differentiated cells provided herein can be administered to a target site or sites on or within a subject in order to replace or compensate for the patient's own damaged, lost, or otherwise dysfunctional cells. This includes infusion of the cells into the patient's bloodstream. The cells to be administered can be cells of the same cell type as those damaged, lost, or otherwise dysfunctional, or a different cell type.

The cells provided herein can be administered as autografts, syngeneic grafts, allografts, and xenografts, for example. As used herein, the term “graft” refers to one or more cells intended for implantation within a human or other animal. Hence, the graft can be a cellular or tissue graft, for example.

Differentiated cells provided herein, or stem cells that are induced to differentiate using the methods provided herein, can be administered to a patient by any method of delivery, such as intravascularly, intracranially, intracerebrally, intramuscularly, intradermally, intravenously, intraocularly, orally, nasally, topically, or by open surgical procedure, depending upon the anatomical site or sites to which the cells are to be delivered. For example, differentiated cells can be administered to the brain during stereotactic surgery, or by intravascular interventional methods using catheters going to the blood supply of the specific organs, or by interventional methods such as intrahepatic artery injection of pancreatic cells for diabetics.

The cells produced using a method provided herein can be administered to a subject in isolation or within a pharmaceutical composition comprising the cells and a pharmaceutically acceptable carrier. As used herein, a pharmaceutically acceptable carrier includes solvents, dispersion media, coatings, antibacterial and antifungal agents, isotonic agents, and the like. Pharmaceutical compositions can be formulated according to known methods for preparing pharmaceutically useful compositions. Formulations are described in a number of sources that are well known and readily available to those of ordinary skill in the art. For example, Remington's Pharmaceutical Science (Martin E. W., Easton Pa., Mack Publishing Company, 19^{th} ed.) describes formulations that can be used in connection with the subject invention. Formulations suitable for parenteral administration, for example, include aqueous sterile injection solutions, which may contain antioxidants, buffers, bacteriostats, and solutes that render the formulation isotonic with the blood of the intended recipient; and aqueous and nonaqueous sterile suspensions that may include suspending agents and thickening agents. It should be understood that in addition to the ingredients particularly mentioned above, the formulations of the subject invention can include other agents conventional in the art having regard to the type of formulation and route of administration in question.

The cells provided herein can be administered on or within a variety of carriers that can be formulated as a solid, liquid, semi-solid, etc. For example, genetically modified cells or non-genetically modified cells can be suspended within an injectable hydrogel composition (U.S. Pat. No. 6,129,761) or encapsulated within microparticles (e.g., microcapsules) that are administered to the patient and, optionally, released at the target anatomical site (Read T. A. et al., Nature Biotechnology, 2001, 19:29-34, 2001; Joki T. et al., Nature Biotechnology, 2001, 19:35-38; Bergers G. and Hanahan D., Nature Biotechnology, 2001, 19:20-21; Dove A. Nature Biotechnology, 2002, 20:339-343; Sarkis R. Cell Transplantation, 2001, 10:601-607).

Carriers are preferably biocompatible and optionally biodegradable. Suitable carriers include controlled release systems wherein the cells and/or the biological factors produced by the cells are released from the carrier at the target anatomic site or sites in a controlled release fashion. The mechanism of release can include degradation of the carrier due to pH conditions, temperature, or endogenous or exogenous enzymes, for example.

The cells provided herein can be administered in or on various scaffolds, such as synthetic or biological tissue scaffolds (Griffith G. and Naughton G., Science, 2002, 295:1009-1013; Langer R., Stem Cell Research News, Apr. 1, 2002, pp. 2-3). Porous scaffold constructs can be composed of a variety of natural and synthetic matrices, such as biominerals (e.g., calcium phosphate) and polymers (e.g., alginate) that are optionally cross-linked, and serve as a template for cell proliferation and ultimately tissue formation. Three-dimensional control of pore size and morphology, mechanical properties, degradation and resorption kinetics, and surface topography of the scaffold can be optimized for controlling cellular colonization rates and organization within an engineered scaffold/tissue construct. In this way, the morphology and properties of the scaffold can be engineered to provide control of the distribution of bioactive agents (e.g., proteins, peptides, etc.) and cells. In addition to use as vehicles for delivery of the proliferated cells, scaffolds can be utilized to grow the cells in vitro. Optionally, cells can be proliferated on the scaffolds themselves using the methods of the subject invention.

The cells provided herein are preferably administered to a subject in an amount effective to provide a therapeutic benefit. A “therapeutically effective amount” is that amount effective to treat a pathological condition. For purposes of the subject invention, the terms “treat” or “treatment” include preventing, inhibiting, reducing the occurrence of and/or ameliorating the physiological effects of the pathological condition to be treated. Preferably, the cells are administered to the patient in an amount within the range of about 10⁴ to about 10¹⁰ cells. It will be understood that doses of cells can be determined with consideration given to such factors as cell survival rate following administration, the number of cells necessary to induce a physiologic response in the normal state, and the species of the subject.

The methods provided herein contemplate intracerebral grafting of differentiated or differentiating cells to a region of the CNS, such as a region having sustained defect, disease, or trauma. Neural transplantation or “grafting” involves transplantation of cells into the central nervous system or into the ventricular cavities, or subdurally onto the surface of the host brain. Conditions relevant to successful transplantation include: (i) viability of the implant; (ii) retention of the graft at the appropriate site of transplantation; (iii) minimum amount of pathological reaction at the site of transplantation; (iv) maintenance of specific cell function; (v) prevention of immune reaction; and (vi) provision of trophic support and vascular supply. Parameters relevant to the above conditions include source of tissue, donor age, number of donors, distribution of grafted tissue, site of implantation, method of cell storage, and type of graft (cell suspension or solid).

Methods for transplanting various nerve tissues as allografts and xenografts are known (See e.g., Freeman T. B. et al., Progress in Brain Research, 1988, Chapter 61, 78:473-477; Freeman T. B. et al., Parkinson's Disease: Advances in Neurology, 2001, Chapter 46, 86:435-445; Freeman T. B. et al., Annals of Neurology, 1995, 38(3):379-387; Freeman T. B. et al., Progress in Brain Research, 2000, Chapter 18, 127:405-411; Olanow C. W. et al. The Basal Ganglia and New Surgical Approaches for Parkinson's Disease, Advances in Neurology, 1997, 74:249-269; Bjorklund et al., Neural Grafting in the Mammalian CNS, 1985, p. 709, Elsevier, Amsterdam; Das G. D., Neural Grafting in the Mammalian CNS, 1985, Chapter 3, p. 23-30, Elsevier, Amsterdam). These procedures include intraparenchymal transplantation, i.e., within the host brain tissue (as compared to outside the brain or extraparenchymal transplantation) achieved by injection or deposition of tissue within the host brain so as to be opposed to the brain parenchyma at the time of transplantation.

Methods for intraparenchymal transplantation include, for example: (i) injecting the donor cells within the host brain parenchyma (e.g., stereotactically, using image guidance, and/or with a catheter attached to a pump, such as a MEDTRONIC system); and (ii) preparing a cavity by surgical means to expose the host brain parenchyma and then depositing the graft into the cavity. Such methods provide parenchymal apposition between the graft and host brain tissue at the time of grafting, and both facilitate anatomical integration between the graft and host brain tissue.

Alternatively, the graft can be place into the cerebral spinal fluid (CSF), either by open surgical injection, intraventricularly via a needle or ventricular reservoir, into the lumbar subarachnoid space using a lumbar puncture, or into any CSF site using a pump and a catheter (e.g., MEDTRONIC). These methods would lend themselves to repeated administration over time, to the CSF or to the brain. Grafting to the ventricle may be accomplished by injection of the donor cells or by growing the cells in a substrate such as 3% collagen to form a plug of solid tissue which may then be implanted into the ventricle to prevent dislocation of the graft. For subdural grafting, the cells may be injected around the surface of the brain after making a slit in the dura. Injections into selected regions of the host brain can be made by drilling a hole and piercing the dura to permit the needle of a microsyringe to be inserted. The microsyringe can be mounted in a stereotactic frame and three-dimensional stereotactic coordinates are selected for placing the needle into the desired location of the brain or spinal cord. Image guidance methods can also be utilized. The cells of the subject invention can also be introduced into the putamen, caudate nucleus, pallidum, nucleus basalis, hippocampus, cortex, cerebellum, subcortical white matter, other regions of the brain, as well as the spinal cord using intravascular technique (Amar A. P. et al. Neurosurgery [2003] 52:402-413).

Smith-Lemli-Opitz syndrome (SLOS), desmosterolosis and lathosterolosis are human syndromes caused by defects in the final stages of cholesterol biosynthesis. Many of the developmental malformations in these syndromes occur in tissues and structures whose embryonic patterning depends on signaling by the Hedgehog (Hh) family of secreted proteins. The Example provided herein illustrates that response to the Hh signal is compromised in mutant cells from mouse models of SLOS and lathosterolosis and in normal cells pharmacologically depleted of sterols. It is shown that decreasing levels of cellular sterols correlate with diminishing responsiveness to the Hh signal. This diminished response occurs at sterol levels sufficient for normal autoprocessing of Hh protein, which requires cholesterol as cofactor and covalent adduct. It is further found that sterol depletion affects the activity of Smoothened (Smo), an essential component of the Hh signal transduction apparatus.

Since normal cells pharmaceutically depleted of sterols appear similar to cells from patients afflicted with SLOS, desmosterolosis, and lathosterolosis, in having a compromised Hh response pathway due to reduced sterol levels, in vitro methods provided herein for differentiating stem cells or progenitor cells can be used in screening methods to identify test compounds that are effective for treating disorders involving defective sterol biosynthesis such as SLOS, desmosterolosis, and lathosterolosis.

Accordingly, provided herein In yet another embodiment, provided herein is a method for identifying a test compound that restores responsiveness to a Hedgehog (Hh) signal, including contacting a cell with a Hh protein, β-cyclodextrin (βCD), under conditions sufficient to decrease sterol concentrations in the cell; and a test compound. Test compounds that restore responsiveness to an Hh signal can be identified by identifying test compounds that stimulate a higher level of responsiveness to the Hh signal as compared with the level of responsiveness in the absence of the test compound.

The cell, such as an isolated cell, is typically a population of cells. The population of cells can be activated in a substantially uniform manner. The method generally is used as a screening assay to identify previously unknown molecules that can restore an Hh response to a cell whose response is comprised by reduced sterol levels. Sterol levels in such cells are reduced in illustrative embodiments to levels sufficient to inhibit responsiveness of the cell to an Hh signal, while providing sufficient sterols to allow Hh autoprocessing and Hh signaling, as discussed in more detail herein. It will be understood that a target reduction in sterol levels is cell type dependent. As illustrated herein in primary fibroblasts, decrease in sterols by about 25% to about 30 μg/ml is sufficient to block responsiveness to an Hh signal.

The Hh protein can be any Hh protein as disclosed herein, such as an ShhN polypeptide, as disclosed herein for other embodiments of the invention. The Hh protein is typically used at a concentration and under conditions known to stimulate a Hh response in the absence of βCD, as illustrated in the Example herein. Methods are known and illustrated in the Example herein, for identifying an Hh response. For example, expression of a reporter gene under the regulation of an Hh responsive regulatory element, such as Gli, can be detected, as illustrated herein.

The cell can be any cell known to respond to an Hh signal, including an isolated cell. For example, the cell can be a fibroblast, such as isolated from a cell line, or a primary fibroblast, as exemplified herein. In certain aspects, the cell lacks 7-hydrocholesterol reductase (Dhcr7) or lathosterol 5-desaturase (Sc5d) enzymes, or is a cell from a human subject afflicted with SLOS or lathosterolosis.

In certain aspects, the isolated cell is a stem cell or a progenitor cell, wherein a test compound is screened for the ability to affect changes in an Hh response genes and/or neural differentiation in the cells contacted with βCD. The stem cell or a progenitor cell can be, for example, a neural plate explant cell. For examples a chicken neural plate explant cell. In other embodiments, the cell is a mammalian cell, such as a human cell.

In another embodiment, a method of screening a compound for neuroactivity is provided. The method includes contacting a stem cell with a Hedgehog protein and β-cyclodextrin (βCD) under conditions sufficient to decrease sterol concentration in the cell, thereby inducing the stem cell to differentiate into a neuron; contacting the neuron with a test compound; and detecting an effect of the test compound on the neuron. A test compound that effects the neuron is neuroactive. In certain illustrative aspects, the Hedgehog protein is a Sonic Hedgehog protein.

Neuroactive drugs which act similarly to those already known to affect neuronal cells can thus be identified. For example, new drugs that alleviate anxiety, analogously to Valium, which augment or stimulate the action of the important inhibitory transmitter gamma-aminobutyric acid (GABA), can be identified. Antidepressants, such as Prozac, enhance the action of serotonin, an indoleamine with a wide variety of functions. Other drugs can be readily identified using the neurons produced herein according to the method of the invention. Other examples include psychoactive compounds. For example, cocaine facilitates the action of dopamine, whereas certain antipsychotics antagonize or inhibit this catecholamine. Another example is nicotine which activates the acetylcholine receptors which are distributed throughout the cerebral cortex. In another aspect, the effect detected can be, for example, a change in gene expression in the neuron. Therefore, by using neuronal cells, drugs and trophic factors which bind various receptors and produce effects on neuronal cells can be identified using the method of the invention.

In another embodiment, provided herein is a method to identify a test compound that affects neuronal differentiation, including contacting a stem cell or a progenitor cell with the following: i) a Sonic Hedgehog protein, ii) cyclodextrin (CD), under conditions sufficient to decrease sterol concentrations in the cell; and iii) a test compound, under conditions sufficient to cause the stem cell or the progenitor cell to differentiate into a neuron in the absence or presence of the test compound. Test compounds that affect neuronal differentiation are identified by identifying test compounds that affect, for example compounds that accelerate or inhibit differentiation of the stem cell or the progenitor cell into a neuron. A test compound that accelerates differentiation is useful, for example, for treating cellular proliferative disorders of neurological tissue.

The test compound, βCD, and the Hh protein in certain embodiments, can be contacted in any order as desired, although typically the cells are continuously exposed to βCD after exposure to the test compound and optionally Hh protein. In certain examples cells are exposed to βCD before exposure to the test compound and the Hh protein. As illustrated in the Examples herein, instead of continuous exposure to βCD, the cells can be exposed to another sterol disrupting agent such as compactin, after transient exposure to βCD. As such, the screening method can be used to identify agents that can overcome the inhibition of responsiveness to an Hh signal by βCD.

The term “test compound” or “test molecule” is used broadly herein to mean any agent that is being examined for agonist or antagonist activity in a method of the invention. A screening method of the invention can use molecular modeling to identify candidate agents. The utilization of a molecular modeling method provides a convenient, cost effective means to identify those agents, among a large population such as a combinatorial library of potential agents, that are most likely to affect an Hh response, thereby reducing the number of potential agents that need to be screened using an assay.

A screening method of the invention provides the advantage that it can be adapted to high throughput analysis and, therefore, can be used to screen combinatorial libraries of test compounds in order to identify those agents that can modulate binding of the first molecule to the second molecule. Methods for preparing a combinatorial library of molecules that can be tested for a desired activity are well known in the art and include, for example, methods of making a phage display library of peptides, which can be constrained peptides (see, for example, U.S. Pat. No. 5,622,699; U.S. Pat. No. 5,206,347; Scott and Smith, Science 249:386-390, 1992; Markland et al., Gene 109:13 19, 1991; each of which is incorporated herein by reference); a peptide library (U.S. Pat. No. 5,264,563, which is incorporated herein by reference); a peptidomimetic library (Blondelle et al., Trends Anal. Chem. 14:83 92, 1995; a nucleic acid library (O'Connell et al., supra, 1996; Tuerk and Gold, supra, 1990; Gold et al., supra, 1995; each of which is incorporated herein by reference); an oligosaccharide library (York et al., Carb. Res., 285:99 128, 1996; Liang et al., Science, 274:1520 1522, 1996; Ding et al., Adv. Expt. Med. Biol., 376:261 269, 1995; each of which is incorporated herein by reference); a lipoprotein library (de Kruif et al., FEBS Lett., 399:232 236, 1996, which is incorporated herein by reference); a glycoprotein or glycolipid library (Karaoglu et al., J. Cell Biol., 130:567 577, 1995, which is incorporated herein by reference); or a chemical library containing, for example, drugs or other pharmaceutical agents (Gordon et al., J. Med. Chem., 37:1385-1401, 1994; Ecker and Crooke, Bio/Technology, 13:351-360, 1995; each of which is incorporated herein by reference).

In another embodiment, the present invention provides valuable research tools for identifying cellular processing involved in neural differentiation, since differentiation using the methods provided herein occurs in a substantially uniform manner. Therefore, samples obtained from cells used in the methods provided herein, such as nucleic acid samples or protein samples, are obtained from cells of a substantially uniform differentiation state.

Accordingly, provided herein, is a method to identify a gene involved in neuronal differentiation, including contacting a stem cell or a progenitor cell with a Sonic Hedgehog protein, under conditions sufficient to cause the stem cell or the progenitor cell to differentiate into a neuron; and cyclodextrin (CD), under conditions sufficient to decrease sterol concentrations in the cell. A gene involved in neuronal differentiation is detected by detecting a gene whose expression changes during differentiation of the stem cell or the progenitor cell.

In another embodiment, provided herein is a method of diagnosing a neurological disorder of a subject, including detecting reduced sterol levels or a reduced response to a Hedgehog signal ni cells of the subject. The method can detect disorders associated with defects in sterol biosynthesis, such as cholesterol biosynthesis. Furthermore, the disorder can be detected by detecting a reduced responsiveness to a Hedgehog signal. The neurological disorder, in certain illustrative examples is Smith-Lemli-Optz syndrome (SLOS), desmosterolosis, or lathosterolosis.

The following examples are intended to illustrate but not limit the invention.

### EXAMPLE 1

### A Defective Response to Hedgehog Signaling in Disorders of Cholesterol Biosynthesis

This example illustrates that response to the Hh signal is compromised in mutant cells from mouse models of SLOS and lathosterolosis and in normal cells pharmacologically depleted of sterols. It is shown that decreasing levels of cellular sterols correlate with diminishing responsiveness to the Hh signal. This diminished response occurs at sterol levels sufficient for normal autoprocessing of Hh protein, which requires cholesterol as cofactor and covalent adduct. It is further found that sterol depletion affects the activity of Smoothened (Smo), an essential component of the Hh signal transduction apparatus. Finally, it is illustrated that sterol depletion in vitro using CD can be used to produce populations of uniformly differentiated neurons in response to a Hh signal.

Methods

Chick embryos. Methyl-β-cyclodextrin (200 μl of a 10% w/v solution in L-15 medium (Sigma and Life Technologies)) was applied to windowed, fertile chick eggs (White Leghorn) after 15 h of incubation. For an average egg volume of 50 ml, the final concentration of cyclodextrin was 375 μM. After 4 d of further incubation, we processed the embryos for scanning electron microscopy. The neural plate and epidermal ectoderm was dissected from stage 9-10 chick embryos, cultured them in collagen and induced them with purified Sonic Hh N-terminal peptide ShbN²¹ as described³. Methyl-β-cyclodextrin was added to the chick explant cultures 4 h after induction with ShhN was initiated.

Analysis of Shh protein biogenesis. MEFs were plated in a 10-cm² dish (Falcon) and transfected them (Fugene 6, Roche) with a full length Shh expression construct (pRK5-Shh, 5 μg) when the cells were roughly 75% confluent. The next day, we changed the culture medium (Dulbecco's modified Eagle medium with 10% fetal bovine serum) to contain either 0.5% fetal bovine serum, 0.5% lipid-depleted serum or lipid-depleted serum after a 30-min treatment with 3.8 mM methyl-β-cyclodextrin (Sigma). After an additional 24 h in culture, we lysed the MEFs in RIPA buffer (50 mM Tris-C1, pH 7.5, 150 mM NaCl, 1% Nonidet P-40, 0.5% sodium deoxycholate, 1 mM EDTA, 1 μg ml⁻¹ leupeptin, 1 μg ml aprotinin⁻¹, 0.2 mM phenylmethylsulfonyl fluoride), immunoprecipitated Shh protein from the cell lysates with a monoclonal antibody that recognizes the N-terminal signaling domain (5E1, Developmental Studies Hybridoma Bank) and immunoblotted them with a polyclonal antibody preparation (JH134).

Shh signaling assays. Shh signaling assays were carried out as described¹⁶ in primary fibroblasts lacking functional Dhcr7, Sc5d or Ptch and in NIH3T3 fibroblasts. Dhcr7^{-/-} and Dhcr7^{+/-} MEFs were generated from embryonic day-9 mutant mice and Sc5h1 and Sc5d^{-/-} and Sc5d^{+/-} MEFs from embryonic day-14.5 mutant mice. We generated a reporter construct with firefly luciferase and Gli (pGL3-8xGli-luciferase) by cloning eight tandem Gli-binding sites and a lens crystallin promoter from the 8xGli-BS Luc construct²² into the pGL3-Basic vector (Promega). We determined relative Hh pathway activity in Dhcr7^{-/-}, Dhcr7^{+/-}, Sc5d^{+/+} and Sc5d^{+/-} MEFs from the expression of transiently transfected pGL3-8xGli-luciferase and control Renilla luciferase (pRL-SV40; Promega) vectors. In Ptch^{-/-} MEFs, we normalized β-galactosidase activity expressed under the control of the Ptch promoter for protein levels to determine relative Hh pathway activity. In the NIH3T3 cell clone Shh-LIGHT Z3, we normalized 8xGli-BS luciferase activity for β-galactosidase activity from stably transfected vectors (8xGli-BS Luc²² and piZ-lacZ). We established clonal sublines of Shh-LIGHT Z3 by cotransfecting either Smo tagged with Myc epitope or SmoA1 with vector encoding G418 resistance (pGT; Invivogen). All transfections were done with Fugene 6. We carried out cyclodextrin treatments for 30 min with methyl-β-cyclodextrin (Sigma) dissolved in Dulbecco's modified Eagle medium, preceded and followed by two washes with phosphate-buffered saline. Fetal bovine serum was depleted of lipids as described²³. We purchased compactin as an active sodium salt form of mevastatin (Biomol) and dissolved it in dimethylsulfoxide.

Sterol analysis. Neutral sterols were extracted and analyzed as described²⁴ from replicate wells of MEFs cultured in parallel with those used for signaling assays.

Results

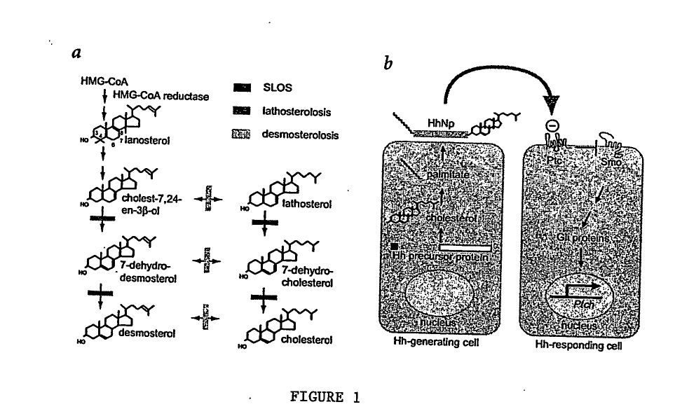

FIGS. 1a-1b diagrammatically illustrate cholesterol biosynthesis and an Hh pathway. a, Autosomal recessive disorders with multiple developmental anomalies are associated with three different enzymatic defects (represented by solid gray-shaded vertical lines) in the final steps of the cholesterol biosynthesis pathway. SLOS results from defects in DHCR7 (dark gray shaded line) (blocking conversion of the 7-dehydrosterol to the sterol, lathosterolosis from defects in SCSD (medium gray shaded line), and desmosterolosis from defects in 3-β-hydroxysterol-Δ²⁴-reductase (light gray shaded line). Steps in sterol synthesis not shown are indicated by multiple arrows. b, After cleavage of the signal sequence (black box), the Hh precursor undergoes an internal cleavage reaction mediated by sequences in the C-terminal autoprocessing domain (white box). Cholesterol participates in the reaction and remains esterified to the newly formed C terminus of the signaling domain (shaded box). Fully processed, secreted Hh proteins (designated HhNp, p for processed) also receive an N-terminal palmitoyl group in a reaction requiring the acyltransferase Skinny hedgehog³⁰. The response to Hh is regulated by two transmembrane proteins, Ptch and Smo. Ptch suppresses the activity of Smo, and Hh binding to Ptch inhibits this function (−), leading to Smo activation of a transcriptional response through the Gli family of transcription factors. Ptch is a transcriptional target of Hh signaling and thus forms a negative feedback loop that ensures adequate regulation of Smo in the absence of Hh. Multiple arrows indicate the participation of a complex of Hh signaling components not shown.

The role of cholesterol in Hh protein biogenesis suggested that impaired Hh autoprocessing might underlie some of the developmental abnormalities in Smith-Lemli-Opitz syndrome (SLOS) (FIG. 1 and Table 1; ref. 1). An additional role for cholesterol in Hh signal response was suggested by the observation that cyclopamine and jervine, teratogenic plant alkaloids that block Hh signaling, also inhibit cholesterol transport and synthesis. But cyclopamine has since been shown to specifically inhibit Hh signaling by binding to a pathway component, and the doses of these alkaloids required to inhibit Hh signaling are lower than those required to block cholesterol transport (ref. 5).

To determine how cholesterol may affect Hh signaling in embryonic development, chick embryos were exposed to cyclodextrin, a cyclic oligosaccharide that forms non-covalent complexes with sterols and can be used to extract and deplete cholesterol from living cells. Cyclodextrin treatment caused variable loss of the frontonasal process and other midline structures (FIG. 2a), and the spectrum of facial defects was similar to that resulting from exposure to the Hh-pathway antagonist jervine. The most severely affected embryos developed a proboscis-like structure that phenocopies the nasal rudiments of mouse embryos that are homozygous with respect to mutations in the gene Sonic hedgehog (Shh; ref. 8).

FIG. 2a provides scanning electron micrographs of facial features at embryonic day 5 of a control (control) chick embryo and of embryos exposed to 375 μM cyclodextrin (CD). Cyclodextrin treatment at the early-primitive-streak stage led to variable loss of midline tissues, primarily the frontonasal process (fnp), and approximation or fusion of paired lateral structures, such as the lateral nasal processes (lnp), optic vesicles (opt) and the maxillary (mx) and mandibular (mn) processes. The slanting nostrils of less severely affected chick embryos treated with cyclodextrin resembled the anteverted nares characteristic of SLOS. Complete loss of the frontonasal process and subsequent fusion of the lateral nasal processes (marked by the asterisk at the top border of the lateral nasal process) led to the formation of a proboscis with a single nasal pit, positioned above fused optic vesicles.

TABLE 1
Developmental malformations in tissues patterned by Hh signaling

| Hh family | Source of | | Normal role of | Consequence of | | | | |
|---|---|---|---|---|---|---|---|---|
| member | Hh signal | Target tissue | Hh signaling | disrupted Hh signaling | PHS²⁵ | SLOS²⁵ | Lath²⁶ | Des²⁷ |
| | Prechordal plate | Ventral | Formation of | Holoprosencephaly | + | + | − | + |
| | mesendoderm and | neuroectoderm and | midline forebrain | | | | | |
| | ectoderm of facial | mesenchyme of | and facial structures | | | | | |
| | processes | facial processes | | | | | | |
| Sonic | Posterior limb bud | Limb bud | Patterning of the | Post-axial polydactyly | + | + | + | |
| hedgehog | mesenchyme | mesenchyme and | autopod and | and syndactyly | + | + | + | |
| | | ectoderm | zeugopod | | | | | |
| | Lung bud | Lung bud | Branching | Unilobular lungs | + | + | | |
| | epithelium | mesoderm | morphogenesis of | | | | | |
| | | | lung | | | | | |
| | Prehypertrophic | Chondrocytes and | Regulation of | Rhizomelia | + | + | | |
| | chondrocytes | osteoblasts | endochondral bone | | | | | |
| | | | growth | | | | | |
| Indian | Colonic epithelium | Neural crest cells | Development of the | Aganglionic | + | + | | |
| hedgehog | | (?) | enteric nervous | megacolon | | | | |
| | | | system | (Hirschsprung disease) | | | | |
| Desert | Sertoli cells | Leydig cells | Development of the | Cryiptorchidism/ | +/+ | +/+ | +/+ | +/+ |
| hedgehog | | | male gonad | ambiguous genitalia or | | | | |
| | | | | hypospadias | | | | |

Overlap of developmental anomalies in tissues patterned by members of the mammalian family of Hh signaling proteins²⁸ among individuals with Pallister-Hall syndrome (PHS), SLOS, lathosterolosis (Lath) and desmosterolosis (Des). PHS is an autosomal dominant disorder associated with mutations in GLI3 that reduce transcriptional activation of Hh pathway targets.²⁹

It was further investigated whether SSH signaling in embryonic tissues by exposing chick neural-plate explants to recombinant Shh protein (ShhN, 30 nm) in the presence of cyclodextrin (FIG. 2b). FIG. 2b provides confocal microscope images of stage 9-10 chick embryo ectoderm dissected from a region of the neural plate intermediate to the notochord and roof plate and just rostral to Hensen's node. Explants were cultured for 48 h in collagen and stained for HNF3β or lSL1, as indicated. Neural progenitor cells explanted from this position and at this developmental stage did not express markers of either floor-plate (HNF3β or motor-neuron (lSL1) cell fates. Addition of 30 nM ShhN at the onset of culture induced a high-threshold response indicated by uniform production of HNF3β (hepatocyte nuclear factor 3β or forkhead box A2; ref. 3), an indicator of floor plate fate. This high-threshold response was blocked in the presence of 400 μM cyclodextrin and replaced by substantially uniform expression of lSL1, an intermediate-threshold response indicative of motor-neuron fate. At 200 μM cyclodextrin, both HNF3μ and lSL1 were expressed, suggesting that lower levels of cyclodextrin were less inhibitory. As active ShhN protein was exogenously supplied, this dose-dependent inhibition suggests that sterol deficits affect response to the Hh signal.

Furthermore, this effect seemed to be specific, as treatment with 400 μM cyclodextrin alone did not inhibit BMP-induced migration of neural-crest cells⁹ (FIG. 2c). Dorsal neural plate progenitor cells and an endogenous source of BMP, the adjacent epidermal ectoderm, were dissected from a region just rostral to Hensen's node in stage 9-10 chick embryos. After 48 h of culture in the presence of 400 μM cyclodextrin, phase contrast and confocal microscope images show that neural crest-like, HNK1-positive cells migrated from the explant into the surrounding collagen.

Hh protein biogenesis involves internal cleavage and covalent addition of cholesterol through an autoprocessing reaction.¹ Cyclodextrin treatment of cultured cells has previously been reported to interfere with Shh autoprocessing¹⁰, an effect distinct from inhibition of response that we observed. To further investigate whether signal production or signal response is the prevailing inhibitory mechanism in cholesterol synthesis disorders, embryonic fibroblast cell lines were established from mouse models of SLOS¹¹ and lathosterolosis and examined Shh signal biogenesis and response in parallel under identical culture conditions.

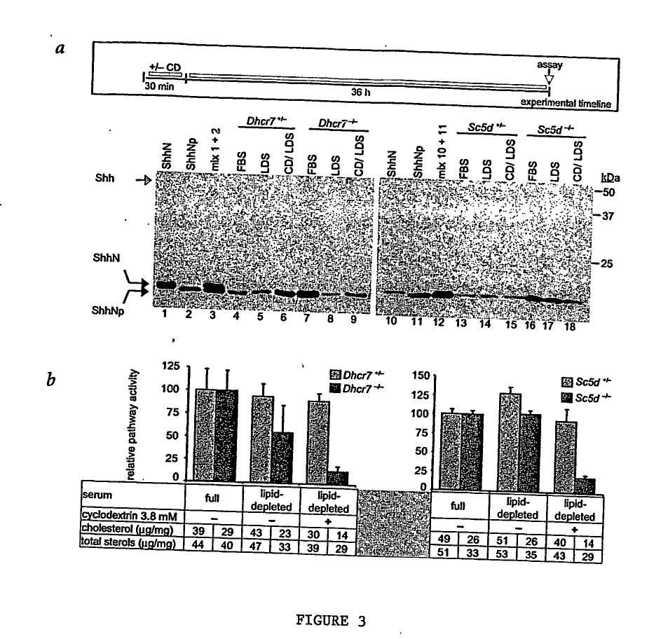

Shh protein was efficiently processed in mouse embryonic fibroblasts (MEFs) lacking 7-dehydrocholesterol reductase (Dhcr7) or lathosterol 5-desaturase (Sc5d) enzymes (models of SLOS and lathosterolosis, respectively; see FIG. 1b), with no observable effect of transient cyclodextrin treatment or growth in lipid-depleted culture medium (FIG. 3a). All of the processed N terminal product (ShhNp) from mutant cultures was cell-associated and had an electrophoretic mobility suggestive of sterol modification (FIG. 3a).

FIGS. 3a-b illustrate that cells defective in cholesterol biosynthesis do not respond to Shh. In FIG. 3a embryonic fibroblasts generated from Dhcr7^{+/-} (lanes 4-6), Dhcr7^{-/-} (lanes 7-9), Sc5d^{+/-} (lanes 13-15) and Sc5d^{-/-} (lanes 16-18) mice were transiently transfected with a full-length Shh expression construct and cultured in full serum (fetal bovine serum, FBS; lanes 4, 7, 13 and 16), lipid-depleted serum (LDS; lanes 5, 8, 14 and 17) or lipid-depleted serum after 30 min of treatment with cyclodextrin (CD/LDS; lanes 6, 9, 15 and 18). Shh was efficiently processed under all culture conditions as there was no detectable accumulation of precursor protein (Mᵣ=45 kDa). Purified ShhNp (lanes 2 and 11; Mᵣ=19.5 kDa) was cell-associated and migrated faster than unprocessed ShhN protein (lanes 1 and 10) collected from the medium of cultured cells transfected with a construct carrying an open reading frame truncated after Gly198 (both ShhNp and ShhN are loaded in lanes 3 and 12), indicating that ShhNp from the treated MEFs (lanes 4-9) probably carried a sterol adduct.

The autoprocessing reaction probably proceeds to completion because cholesterol levels are only reduced by roughly 50% under the conditions used for depletion (FIG. 3b) and because 7-dehydrocholesterol and lathosterol both participate efficiently as sterol adducts in the Shh processing reaction³. Likewise, cholesterol levels are reduced but never absent in the serum of individuals with SLOS¹².

In contrast with their normal Shh autoprocessing, MEFs with mutations in Dhcr7 and Sc5d had clear deficiencies in their ability to respond to Shh signal when transiently treated with cyclodextrin and grown in lipid-depleted culture medium (FIG. 3b). The effect of cholesterol depletion on responsiveness of Dhcr7^{+/-} (gray bars), Dhcr7^{-/-} (maroon bars), Sc5d^{+/-} (gray bars), and Sc5d^{-/-} (green bars) MEFs to Shh signaling was assayed by transfection with a Hh-responsive firefly luciferase reporter and treatment with purified ShhNp (10 nM). MEFs were transfected at sub-confluent densities, cultured to maximum cell density and then induced with ShhNp either in culture medium containing fetal bovine serum (full) or lipid-depleted serum. Some of the cells cultured in lipid-depleted serum were stripped of surface cholesterol with a 30-min exposure to cyclodextrin just before ShhNp induction. For each culture condition, normalized firefly luciferase activity from control and ShhNp treated cells was used to calculate the relative induction, expressed as a percentage of the Shh induction achieved in MEFs cultured in full serum. Bars represent the standard error from quadruplicate experiments.

These results indicate that signal response is more sensitive than is signal biogenesis to perturbations of cholesterol homeostasis. Cells heterozygous with respect to the mutations in Dhcr7 and Sc5d maintained a normal response to ShhNp stimulation under all growth conditions (FIG. 3b), presumably because there was sufficient synthetic activity from the functional allele. In these experiments, the initial transient cyclodextrin treatment is needed to reduce sterol levels to below 40 μg mg⁻¹ protein to affect pathway response (FIG. 3b).

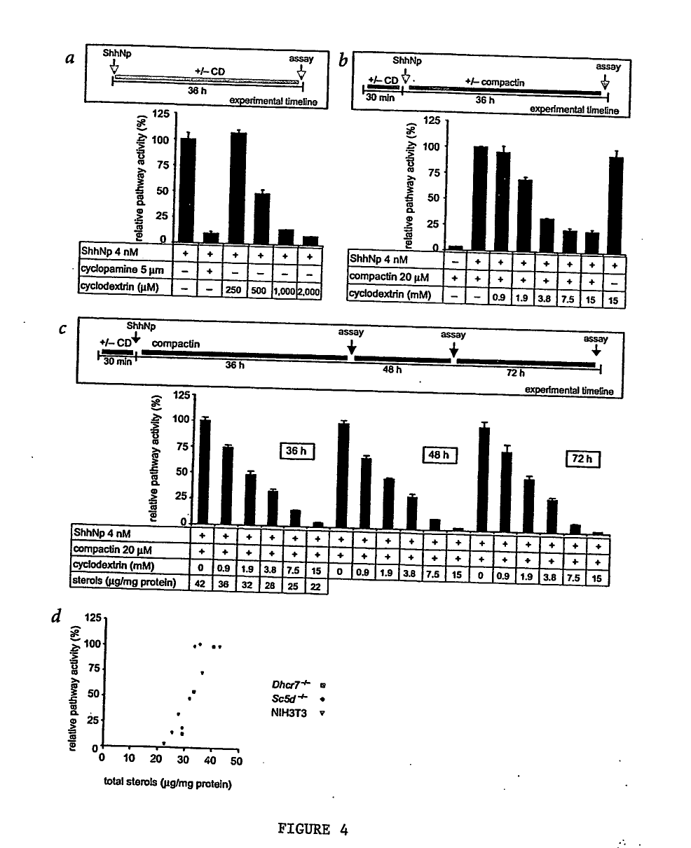

To further investigate Hh signal response in other cultured cells, we tested the ability of pharmacological interventions to mimic the effects of genetic defects in sterol biosynthesis. Continuous treatment with 500 μM cyclodextrin produced about 50% inhibition of Shh signal response in NIH3T3 cells, and 2 mM cyclodextrin produced nearly complete inhibition (FIG. 4a).

FIG. 4a shows NIH3T3 fibroblasts stably transfected with a Hh-responsive luciferase reporter construct (Shh-LIGHT Z3 cells) were chronically depleted of sterols by the addition of cyclodextrin (CD) to the culture medium for the duration of ShhNp induction (see schematic in a). Cyclodextrin treatment inhibited the response to Shh signaling in a dose dependent manner and to a comparable degree as 5 μM cyclopamine¹⁶. FIG. 4b, Shh signal reception was also blocked in Shh-LIGHT Z3 cells by acute sterol depletion with transient exposure to cyclodextrin before ShhNp induction, followed by the addition of an HMG-CoA reductase inhibitor (compactin) during the period of induction (see schematic in b). The combination of transient cyclodextrin treatment followed by inhibition of cholesterol synthesis blocked the response to ShhNp, whereas neither treatment alone did so. FIG. 4c, Hh path way inhibition persisted for 72 h after cyclodextrin treatment. The 3-d recovery period was intended to allow replenishment of non-sterol cellular components depleted by transient cyclodextrin treatment while maintaining sterol depletion with HMG-CoA reductase inhibition by compactin. FIG. 4d, Total cellular sterols from parallel cultures of NIH3T3 (c, 36 h), Dhcr7^{-/-} and Sc5d^{-/-} (FIG. 3b) MEFs were determined by gas chromatography-mass spectrometry analysis of extracted lipids and plotted against the relative Shh pathway activity. Bars represent one standard error (quadruplicate wells).

Transient cyclodextrin treatment for 30 minutes before exposure to ShhNp, however, did not affect response, even at 15 mM cyclodextrin, a concentration 30 times higher than the IC50 for continuous cyclodextrin treatment (FIG. 4b). ShhNp response was also not affected by continuous exposure to compactin, an inhibitor of 3-hydroxy-3-methyl-glutaryl coenzyme A (HMG CoA) reductase that blocks sterol synthesis¹³. The combination of transient cyclodextrin treatment with continuous compactin exposure, however, blocked Shh signal response (FIG. 4b). These data suggest that compactin treatment, when combined with transient cyclodextrin treatment, can mimic a genetic defect in sterol biosynthesis to inhibit Shh signal response. The Shh autoprocessing reaction was not affected by these experimental conditions.

Cyclodextrins form complexes with hydrophobic compounds including proteins and phospholipids in addition to sterols⁶. But the effect of cyclodextrin treatment is probably due to sterol depletion, as transient cyclodextrin treatment only inhibited pathway activity in the presence of sterol biosynthetic mutations or of a statin, and no measurable recovery from the impact of deficits in molecules other than sterols was achieved during prolonged incubation after transient cyclodextrin treatment (FIG. 4c). Furthermore, other sterol-specific perturbations, such as treatment with nystatin or filipin¹⁴, also blocked the response of cells to ShhNp protein (data not shown), and the degree of pathway inhibition in sterol biosynthetic mutant and statin-treated fibroblasts correlated inversely with total sterol levels (FIG. 4d). An inhibitory role for 7-dehydrocholesterol, lathosterol and other cholesterol precursors that accumulate in the Dhcr7 and Sc5d mutant cells seems improbable because synthesis of such precursors in statin-treated wild-type cells is blocked. Indeed, the correlation between responsiveness and overall sterol content (FIG. 4d) suggests that these precursors may contribute to pathway response, although their effectiveness relative to that of cholesterol is unknown.

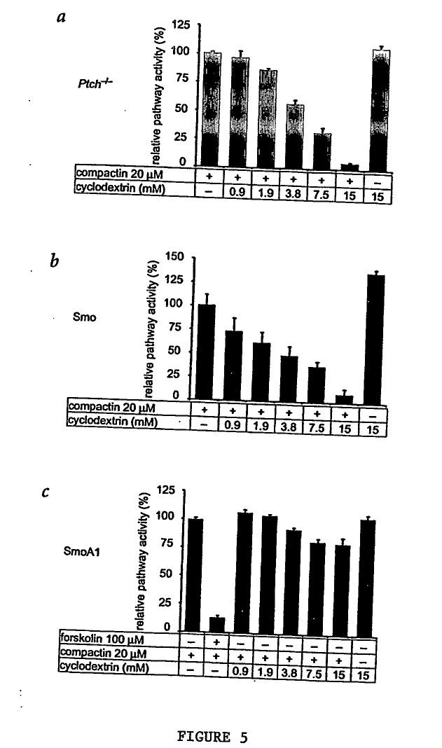

The ability to pharmacologically mimic sterol biosynthetic defects affords an opportunity to identify the Hh pathway component most directly affected by sterol perturbations. Patched (Ptch) and Smoothened (Smo) stand out as candidate targets because of their integral membrane structure and essential roles in regulating cellular response to the Hh signal. Genetic and biochemical evidence indicates that Ptch suppresses the activity of Smo and that Hh binding to Ptch releases this suppression, allowing Smo to activate a transcriptional response through the Gli family of transcription factors (FIG. 1b). We examined the ability of sterol perturbations to affect the constitutive Hh pathway activity in fibroblasts derived from Ptch^{-/-} embryos^{15,16} and found a dose-dependent reduction by transient treatment with increasing concentrations of cyclodextrin in combination with compactin (FIG. 5a). Therefore, sterol depletion can block pathway activity independently of Ptch action and may act at a point in the pathway downstream of Ptch.

FIG. 5a shows the constitutively active Shh pathway in Ptch^{-/-} MEFs (measured as β-galactosidase activity produced from a fusion of lacZ to the third cordon of Ptch¹⁶) was blocked by cholesterol depletion. FIG. 5b, Shh pathway activation by overexpression of wild-type Smo in NIH3T3 cells was also inhibited by cholesterol depletion. But Shh pathway activity driven by overexpression of oncogenic Smo (c; SmoA1, W539L) was resistant to inhibition by cholesterol depletion. Cyclodextrin and compactin treatments for FIGS. 5a-c are as indicated for FIG. 3b. NIH3T3 fibroblasts in b and c were stably transfected with expression constructs for either Srno (b) or activated Smo (SmoA1; c), a Gli-luciferase reporter and lacZ for normalization. Forskolin (100 μM) inhibition of SmoA1-driven pathway activity is illustrated as a positive control¹⁶. Bars represent one standard error from three (a) or six (b,c) replicates for each treatment group.

Next the effects were examined, of depleting sterols in NIH3T3 cells stably transfected to express wild-type or oncogenic, activated Smo (ref. 16; SmoA1; W539L). Sterol depletion was found to completely block the modest level of pathway activation produced by overexpression of Smo (FIG. 5b), but that it scarcely affected pathway activation by SmoA1 (FIG. 5c). The susceptibility of wild-type Smo but resistance of a mutant activated form to sterol depletion suggests that Smo may be the site of sterol action. Consistent with this conclusion, sterol deprivation did not affect the activation of the pathway due to expression of Gli1, which acts downstream of Smo (data not shown). The retention of normal Hh pathway activity in cells expressing activated Smo indicates that pathway components downstream of Smo function normally under conditions of sterol depletion.

Previous work has suggested that Smo activity is governed by a balance between active and inactive conformations¹⁶. The resistance to cholesterol deprivation of activated Smo suggests that Smo con formation may be the target of cholesterol deprivation. This effect could be mediated either through direct interaction of cholesterol with Smo or through an impact on membrane properties, as reported for the function of other seven-transmembrane-domain proteins, such as the oxytocin or the brain cholecystokinin receptors¹⁷. One possibility is that sterol depletion could affect a lipid microdomain or raft-mediated process¹⁸ required for Smo activity, although we did not observe a change in Smo fractionation with respect to detergent-resistant membranes on sterol depletion (data not shown). Alternatively, the effects of sterol depletion on Smo activity might be indirectly mediated through an as yet uncharacterized interacting component.

Sterol depletion has been previously reported to affect Shh auto processing¹⁰. This depletion was probably somewhat more severe than that produced by our experimental manipulations. Furthermore, it was found that Hh signal response is more sensitive than Hh autoprocessing to inhibition by mutational or pharmacological sterol depletion. It is therefore concluded that inhibition of response to Hh protein is a more probable cause of the malformations associated with cholesterol biosynthetic disorders than is inhibition of Hh autoprocessing. Other processes might also be affected by defects in distal cholesterol biosynthesis, as not all of the malformations are necessarily accounted for by impaired Hh signaling. Nevertheless, our findings help explain many developmental malformations associated with a relatively common genetic disorder, SLOS, whose incidence among European Caucasians is 1 in 22,000 (ref 19). Our findings may also be relevant to other etiologies of abnormal human development, as holoprosencephaly is reported to occur at a frequency of 1 in 250 among aborted fetuses²⁰. The surprising connection between cholesterol synthesis and Hh signal response reported here suggests that signaling pathways involved in developmental patterning must be considered as potential targets of any seemingly simple metabolic defect associated with developmental malformations.
1. Porter, J. A., Young, K. E. & Beachy, P. A. Cholesterol modification of hedgehog signaling proteins in animal development. Science 274, 255-259 (1996).
2. Beachy, P. A. et al. Multiple roles of cholesterol in hedgehog protein biogenesis and signaling. Cold Spring Harb. Symp. Quant. Biol. 62, 191-204 (1997).
3. Cooper, M. K., Porter, J. A., Young, K. E. & Beachy, P. A. Teratogen-mediated inhibition of target tissue response to Shh signaling. Science 280, 1603-1607 (1998).
4. Chen, J. K., Taipale, J., cooper, M. D. & Beachy, P. A. Inhibition of Hedgehog signaling by direct binding of cyclopamine to Smoothened. Genes Dev. 16, 2743-2748 (2002).
5. Incardona, J. P. et al. Cyclopamine inhibition of Sonic hedgehog signal transduction is not mediated through effects on cholesterol transport. Dev. Biol. 224, 440-452 (2000).
6. Ohtani, Y., Irie, T., Uekama, K., Fukunaga, K. & Pitha, J. Differential effects of α-, β-, and γ-cyclodextrins on human erythrocytes. Eur. J. Biochem. 186, 17-22 (1989).
7. Kilsdonk, E. P. et al. Cellular cholesterol efflux mediated by cyclodextrins. J. Biol. Chem. 270, 1750-17256 (1995).
8. Chiang, C. et al. Cyclopia and defective axial patterning in mice lacking Sonic hedgehog gene function. Nature 383, 407-413 (1996).
9. Liem, K. F Jr., Tremml, G., Roelink, H. & Jessell, T. M. Dorsal differentiation of neural plate cells induced by BMP-mediated signals from epidermal ectoderm. Cell 82, 969-979 (1995).
10. Guy, R. K. Inhibition of Sonic hedgehog autoprocessing in cultured mammalian cells by sterol deprivation. Proc. Natl. Acad. Sci. USA 97, 7307-7312 (2000).
11. Wassif, C. A. et al. Mutations in the human sterol δ7-reductase gene at 11q12-13 cause Smith-Lemli-Opitz syndrome. Am. J. Hum. Genet. 63, 55-62 (1998).
12. Kelley, R. I. Inborn errors of cholesterol biosynthesis. Adv. Pediatr. 47, 1-53 (2000).
13. Brown, M. S., Fuast, J. R. & Goldstein, J. L. Induction of 3-hydroxy-3-methylglutaryl coenzyme A reductase activity in human fibroblasts incubated with compactin (ML-236B), a competitive inhibitor of the reductase. J. Biol. Chem. 253, 1121-1128 (1978).
14. Bittman, R. & Fischkoff, S. A. Fluorescence studies of the binding of the polyene antibiotics filipin 3, amphotericin B, nystatin, and lagosin to cholesterol. Proc. Natl. Acad. Sci. USA 69, 3795-3799 (1972).
15. Goodrich, L. V., Milkenkovic, L., Higgins, K. M. & Scott, M. P. Altered neural cell fates and medulloblastoma in mouse patched mutants. Science 277, 1109-1113 (1997).
16. Taipale, J. et al. Effects of oncogenic mutations in Smoothened and Patched can be reversed by cyclopamine. Nature 406, 1005-1009 (2000).
17. Gimpl, G., Burger, K. & Fahrenholz, F. Cholesterol as modulator of receptor function. Biochemistry 36, 10959-10974 (1997).
18. Simons, K. & Ikonen, E. Functional rafts in cell membranes. Nature 387, 569-572 (1997).
19. Nawaczyk, M. J., McCaughey, D., Whelan, D. T. & Porter, F. D. Incidence of Smith-Lemli-Opitz syndrome in Ontario, Canada. Am. J. Med. Genet. 102, 18-20 (2001).
20. Matsunaga, E. & Shiota, K. Holoprosencephaly in human embryos: epidemiologic studies of 150 cases. Teratology 16, 261-272 (1977)
21. Roelink, H. et al. Floor plate and motor neuron induction by different concentrations of the amino-terminal cleavage product of Sonic hedgehog autoproteolysis. Cell 81, 445-455 (1995).
22. Sasaki, H., Hui, C., Nakafuka, M. & Kondoh, H. A binding site for Gli proteins is essential for HNF-3β floor plate enhancer activity in transgenics and can respond to Shh in vitro. Development 124, 1313-132 (1997).
23. Gibson, K. M. et al. 3-Hydroxy-3-methylglutaryl coenzyme A reductase activity in cultured fibroblasts from patients with mevalonate kinase deficiency: differential response to lipid supplied by fetal bovine serum in tissue culture medium. J. Lipid Res. 31, 515-521 (1990).
24. Kelley, R. I. Diagnosis of Smith-Lemli-Opitz syndrome by gas chromatography/mass spectrometry of 7-dehydrocholesterol in plasma, amniotic fluid and cultured skin fibroblasts. Clin. Chim. Acta 236, 45-58 (1995).
25. Donnai, D., Burn, J. & Hughes, H. Smith-Lemli-Opitz syndromes: do they include the Pallister-Hall syndrome? Am. J. Med. Genet. 28 741-743 (1987).
26. Parnes, S., Hunter, A. G., Jimenez, C., Carpenter, B. F. & MacDonald, I. Apparent Smith-Lemli-Opitz syndrome in a child with a previously undescribed form of mucolipidosis not involving the neurons. Am. J. Med. Genet. 35, 397-405 (1990).
27. Clayton, P., Mills, K., Keeling, J. & FitzPatrick, D. Desmosterolosis: a new inborn error of cholesterol biosynthesis. Lancet 348, 404 91996).
28. Ingham, P. W. & McMahon, A. P. Hedgehog signaling in animal development: paradigms and principles. Genes Dev. 15, 3059-3087 (2001).
29. Wang, B., Fallon, J. F. & Beachy, P. A. Hedgehog-regulated processing of Gli3 produces an anterior/posterior repressor gradient in the developing vertebrate limb. Cell 100, 423-434 (2000).
30. Chamoun, Z. et al. Skinny hedgehog, an acyltransferase required for palmitoylation and activity of the hedgehog signal. Science 293, 2080-2084 (2001).
31. Cooper M K, Wassif C A, Krakowiak P A, Taipale J, Gong R, Kelley R I, Porter F D, Beachy P A. A defective response to Hedgehog signaling in disorders of cholesterol biosynthesis. Nat Genet. 2003 April; 33(4):508-13. Epub 2003 Mar. 24. Erratum in: Nat Genet. 2003 May; 34(1):113.
32. Di Guglielmo G M, Le Roy C, Goodfellow A F, Wrana J L. Distinct endocytic pathways regulate TGF-beta receptor signalling and turnover. Nat Cell Biol. 2003 May; 5(5):410-21. Erratum in: Nat Cell Biol. 2003 July; 5(7):680.
33. Patten I, Placzek M. Opponent activities of Shh and BMP signaling during floor plate induction in vivo. Curr Biol. 2002 Jan. 8; 12(1):47-52.
34. Liem K F Jr, Jessell T M, Briscoe J. Regulation of the neural patterning activity of Sonic Hedgehog by secreted BMP inhibitors expressed by notochord and somites. Development. 2000 November; 127(22):4855-66.

Although the invention has been described with reference to the above example, it will be understood that modifications and variations are encompassed within the spirit and scope of the invention. Accordingly, the invention is limited only by the following claims.

What is claimed is:

1. A method for inducing differentiation of a stem cell into a neuron, comprising contacting a stem cell with a Hedgehog protein and β-cyclodextrin (βCD) under conditions sufficient to decrease sterol concentration in the cell, thereby inducing the stem cell to differentiate into a neuron.

2. The method of claim 1, wherein the differentiated cell is a motor neuron.

3. The method of claim 1, wherein the differentiated cell is a dopaminergic neuron.

4. The method of claim 1, wherein the stem cell is a mammalian cell.

5. The method of claim 4, wherein the stem cell is a human cell.

6. The method of claim 1, wherein the stem cell is an avian cell.

7. The method of claim 1, wherein the hedgehog is protein is an N-terminal fragment of a hedgehog polypeptide.

8. The method of claim 1, wherein the βCD is β-methyl CD.

9. The method of claim 1, wherein a population of stem cells or are contacted with Hedgehog protein and βCD, wherein the population of stem cell or differentiate into a substantially uniform population of differentiated neurons.

10. The method of claim 1, wherein the βCD is at a concentration effective for reducing sterol levels to below 40 μg/mg.

11. The method of claim 10, wherein the βCD is at a concentration of between about 100 μM and 5 mM.

12. The method of claim 11, wherein the βCD is at a concentration of between about 200 μM and 600 μM.

13. The method of claim 10, wherein the βCD is used at a concentration of about 400 μM.

14. The method of claim 1, wherein the stem cell is a neuronal stem cell.

15. The method of claim 1, wherein the Hedgehog protein is a Sonic Hedgehog protein.

16. The method of claim 15, wherein the ShhN, is an N-terminal fragment of a Sonic Hedgehog protein.

17. A method to alter the responsiveness of a stem cell to a Hedgehog signal, comprising:
a) contacting the stem cell or the progenitor cell with Hedgehog; and
b) contacting the stem cell with βcyclodextrin (βCD) in vitro under conditions sufficient to decrease sterol concentration in the cell, thereby altering the responsiveness to a Hh signal.

18. The method of claim 17, further comprising detecting expression of a Hedgehog responsive gene.

19. The method of claim 17, further comprising detecting expression of a gene whose expression is associated with neuronal differentiation.

20. A method for differentiating a population of stem cells into a population of neurons, comprising: contacting a population of stem cells with a differentiation signaling protein under conditions sufficient to induce differentiation, and a sterol-depleting agent under conditions sufficient to decrease sterol concentrations in the population of cells and/or under conditions sufficient to positively effect TGFβ signaling in the population of cells, wherein the differentiation signaling protein is selected from Hedgehog and a Transforming Growth Factor β (TGFβ) family member, and wherein the population of stem cells differentiate into a substantially uniform population of neurons.

21. The method of claim 20, wherein the sterol-depleting agent is β-cyclodextrin (βCD).

22. The method of claim 21, wherein the stem cell is contacted with βCD under conditions sufficient to positively effect TGFβ signaling.

23. The method of claim 22, wherein the stem cell is contacted with a bone morphogenic protein (BMP).

24. The method of claim 20, wherein the differentiation signaling protein is a Hedgehog protein.

25. The method of claim 24, wherein the differentiation signaling protein is Sonic Hedgehog.

26. The method of claim 25, wherein the population of stem cells is contacted with the sterol-depleting agent under conditions sufficient to decrease sterol concentrations.

27. The method of claim 26, wherein the sterol-depleting agent is cyclodextrin (CD), nystatin, or filipin.

28. The method of claim 24, wherein the differentiation signaling protein is a Hedgehog N-terminal peptide.

29. The method of claim 28, wherein the differentiation signaling protein is Sonic Hedgehog N-terminal peptide.

30. The method of claim 29, wherein the population of stem cells is contacted with jervine or cyclopamine at a concentration lower than a concentration required to block cholesterol transport.

31. A method for differentiating a population of stem cells, comprising:
a) contacting a population of stem cells with a Hedgehog protein under conditions sufficient to induce differentiation; and
b) contacting the population of stem cells with β-cyclodextrin under conditions sufficient to decrease sterol concentration in the cells and/or under conditions sufficient to positively effect TGFβ signaling in the population of cells, wherein the population of stem cells differentiate into a substantially uniform population of differentiated cells.

32. The method of claim 31, wherein the population of stem cells or progenitor cells differentiate into a population of cells selected from cells of the central nervous system, intestinal cells, pancreatic cells, lung cells, and retinal cells.

33. A method of introducing a cell into a subject, comprising:
a) differentiating a stem cell into a differentiated neuron in vitro by the method of claim 1; and
b) introducing the differentiated neuron into the subject.

34. The method of claim 33, wherein a substantially uniform population of differentiated neurons produced by the method of claim 1, are introduced into the subject.

35. The method of claim 34, wherein the subject is a human.

36. The method of claim 35, wherein the human is afflicted with a neurodegenerative disease.

37. A substantially uniform population of differentiated neurons produced by the method of claim 1 or 31.

38. A method of screening a compound for neuroactivity, comprising:
a) contacting a stem cell with a Hedgehog protein and β-cyclodextrin (βCD) under conditions sufficient to decrease sterol concentration in the cell, thereby inducing the stem cell to differentiate into a neuron;
b) contacting the neuron with a test compound; and
b) detecting an effect of the test compound on the neuron, wherein a test compound that affects the neuron is neuroactive.

39. The method of claim 38, wherein the Hedgehog protein is a Sonic Hedgehog protein.

40. The method of claim 38, wherein the effect is a change in gene expression in the neuron.

41. The method of claim 38, wherein the effect on the neuron is selected from augmenting or stimulating of the action of gamma-aminobutyric acid (GABA), enhancing of the action of serotonin, facilitating the action of dopamine, or activating acetylcholine receptors.

42. A method of screening a compound for its effect on neuronal differentiation, comprising:
a) contacting a stem cell with:
i) a Hedgehog protein,
ii) βcyclodextrin (βCD), under conditions sufficient to decrease sterol concentrations in the cell; and
iii) a test compound,
under conditions sufficient to cause the stem cell to differentiate into a neuron in the absence of the test compound; and
b) determining whether the stem cell or the progenitor cell differentiates into a neuron, wherein an absence or presence of differentiation is indicative of a test compound that affects neuronal differentiation.

43. The method of claim 42, wherein stem cell is a neural plate explant cell.

44. The method of claim 42, wherein the cell is a mammalian cell.

45. The method of claim 44, wherein the cell is a human cell.

46. The method of claim 42, wherein the Hedgehog protein is a Sonic Hedgehog protein.

47. A method for identifying an agent that restores responsiveness to a Hedgehog (Hh) signal in a cell with a loss of responsiveness, comprising:
a) contacting the cell with:
i) B-cyclodextrin (BCD), under conditions sufficient to decrease sterol concentrations in the cell;
ii) an Hh protein; and
iii) a test compound; and
b) determining whether the cell is responsive to the Hh signal as compared with the level of responsiveness in the absence of the compound wherein a higher level of responsiveness to the Hh signal in the presence of the compound identifies a compound that restores responsiveness to the Hh signal.

48. The method of claim 47, wherein the isolated cell is a fibroblast.

49. The method of claim 47, wherein the Hh protein is a Sonic Hh protein.

50. A method for identifying a gene involved in neuronal differentiation, comprising:
a) contacting a stem cell with:
i) a Hedgehog (Hh) protein, under conditions sufficient to cause the stem cell or the progenitor cell to differentiate into a neuron; and
ii) B-cyclodextrin (BCD), under conditions sufficient to decrease sterol concentrations in the cell; and
b) detecting a gene whose expression changes following or during differentiation as compared to before differentiation of the stem cell.

51. The method of claim 50, wherein the Hh protein is Sonic Hh protein.

52. The method of claim 51, wherein the Sonic Hh N-terminal peptide.

53. A method for diagnosing a neurological disorder in a subject, comprising detecting reduced sterol levels or a reduced response to a Hedgehog signal in cells of the subject.

54. The method of claim 53, wherein the method detects disorders associated with defects in sterol biosynthesis.

55. The method of claim 54, wherein the disorder is detected by detecting a reduced responsiveness to a Hedgehog signal.

56. The method of claim 54, wherein the defect is in cholesterol biosynthesis.

57. The method of claim 54, wherein the neurological disorder is Smith-Lemli-Optz syndrome (SLOS), desmosterolosis, or lathosterolosis.

58. The method of claim 54, wherein the cells are neurons.
